(19) United States
(12) Patent Application Publication
(10) Pub. No.: US 2023/0314440 A1
(43) Pub. Date: Oct. 5, 2023

(54) METHOD OF TREATING CANCER

(71) Applicant: Exelixis, Inc., Alameda, CA (US)

(72) Inventors: Christopher Del Nagro, Millbrae, CA (US); Jason Wall, Shoreline, WA (US); Evelyn Wang, San Francisco, CA (US); Chih-Jian Lih, Palo Alto, CA (US)

(21) Appl. No.: 18/022,209

(22) PCT Filed: Aug. 20, 2021
(86) PCT No.: PCT/US2021/046949

§ 371 Date: Feb. 20, 2023

Related U.S. Application Data
(60) Provisional application No. 63/068,659, filed on Aug. 21, 2020.

(51) Int. Cl. G01N 33/574

(52) U.S. Cl. CPC G01N 33/57492; G01N 2800/52

(57) ABSTRACT

This invention is directed to the treatment of cancer, particularly solid tumors, using cabozantinib in combination with an immune checkpoint inhibitor or an anti-cancer vaccine and in predicting responses to such cancer treatments.

### CROSS-REFERENCE TO RELATED APPLICATIONS

This application claims priority to U.S. Provisional Application Ser. No. 63/068,659, the entire contents of which are incorporated by reference herein.

### FIELD OF THE INVENTION

This invention is directed to the treatment of cancer, particularly solid tumors, using cabozantinib in combination with an immune checkpoint inhibitor or an anti-cancer vaccine and in predicting responses to such cancer treatments.

### BACKGROUND OF THE INVENTION

As noted by the Immuno-Oncology Working Group, assessment of tumor infiltrating lymphocytes (TILs) in histopathological specimens can provide important prognostic information in diverse solid tumor types. TILs also have value in predicting responses to cancer treatments. Adv. Anat. Pathol. 24; 235-251. In addition, according to the Immuno-Oncology Working Group, the host immune response to tumors is of great interest to oncologists and researchers in light of the promising early results of newer cancer therapies including immune checkpoint inhibitor therapy and anti-cancer vaccines.

The successful clinical use, either alone or combination, of immune checkpoint inhibitors and other forms of immunotherapy such as anti-cancer vaccines has made the need for immuno-oncology biomarkers a reality. The correlation between TIL immune phenotype and objective therapy response is a desirable measure for guiding treatment choices. For example, the kinase inhibitor cabozantinib is now being considered as a cancer treatment in combination with immune checkpoint inhibitors or anti-cancer vaccines. Nature Communications, 2020, 11:2124. J. Tanslational Medicine, 2014, 12:294. Studies have shown that cabozantinib has an impact on systemic tumor immunity including modulating blood immune cell repertoire and influencing systemic adaptive and innate immunity in patients with metastatic renal cell carcinoma (RCC). ESMO 2018, Presentation No. 882P. A synergistic efficacy of a combination of cabozantinib with immune checkpoint blockade was also observed for castration-resistant prostate cancer. Nature, 2017, 543, 728. However, developing better and reliable predictive models for response to immune checkpoint blockade in cancer patients including RCC patients remains a challenge. There is therefore a need to identify immune biomarkers of cabozantinib/immunologic therapies to optimize treatment.

### SUMMARY OF THE INVENTION

These and other needs are met by the present invention, which is directed to the identification and measurement of TILs in patients undergoing cancer treatment alone or in combination with one or more immune checkpoint inhibitors.

The Applicant surprisingly and unexpectedly found that when treated with cabozantinib in combination with an immune checkpoint inhibitor, for example, atezolizumab, patients with T cell rich tumors characterized by high levels of myeloid (CD68 High) and cytotoxic T cells (CD8 High) showed the greatest level of tumor shrinkage from baseline and overall response compared to CD68 High/CD8 Low and CD68 Low/CD8 Low phenotypes. The CD68 Low/CD8 High phenotype was not observed. It was also surprising to find that PD-L1 positive immune cells and/or high frequency of CD8 T cells are significantly associated with overall response and lesion change in RCC patients treated with cabozantinib in combination with an immune checkpoint inhibitor, for example, atezolizumab. These results suggest the significant role TILs can play in guiding treatment with cabozantinib, either alone or in combination with other agents such as PD-1 or PD-L1 inhibitors.

Thus, in one aspect, the disclosure relates to a method for predicting a treatment response of a subject having cancer to a therapy comprising cabozantinib in combination with an immune checkpoint inhibitor or a cancer vaccine, the method comprising:
a. measuring the myeloid cell and T cell populations in a tumor tissue sample from the subject; and
b. associating with the treatment response of the subject based on the relative abundance/percentage of myeloid cell population and T cell population.

In another aspect, the disclosure relates to a method for identifying a subject having cancer as a candidate for treatment with a therapy comprising cabozantinib in combination with an immune checkpoint inhibitor or a cancer vaccine, the method comprising:

analyzing the myeloid cell and T cell populations in a tumor tissue sample from the subject, wherein the relative abundance/percentage of myeloid cell population and T cell population indicates the subject may benefit from the treatment with cabozantinib in combination with an immune checkpoint inhibitor or a cancer vaccine.

In a further aspect, the disclosure relates to a method for identifying a subject having cancer that has an increased benefit from a treatment comprising cabozantinib in combination with an immune checkpoint inhibitor or a cancer vaccine, the method comprising:

analyzing the myeloid cell and T cell populations in a tumor tissue sample from the subject, wherein the relative abundance/percentage of myeloid cell population and T cell population is indicative of the subject may benefit from the treatment.

In a further aspect, the disclosure relates to a method of treating cancer in a subject, the method comprising:
analyzing the myeloid cell and T cell populations in a tumor tissue sample from the subject, wherein the relative abundance/percentage of myeloid cell population and T cell population is indicative of the subject may benefit from the treatment; and
treating the subject in need of the treatment with a combination therapy comprising cabozantinib in combination with an immune checkpoint inhibitor or a cancer vaccine.

In another aspect, the disclosure relates to a method of treating a subject having a solid tumor with cabozantinib in combination with an immune checkpoint inhibitor therapy, comprising:
a. determining whether the solid tumor comprises myeloid CD68 cells and cytotoxic CD8 T-cells; and
b administering one or more doses of cabozantinib and one or more doses of immune checkpoint inhibitor therapy to the subject if the tumor comprises myeloid CD68 cells and cytotoxic CD8 T-cells, wherein the presence of myeloid CD68 cells and cytotoxic CD8 T-cells in the tumor indicates the responsiveness of the tumor to the treatment with the checkpoint inhibitor therapy.

In another aspect, the disclosure relates to a method for predicting a treatment response of a subject having cancer to a therapy comprising cabozantinib in combination with an immune checkpoint inhibitor or a cancer vaccine, the method comprising:
a. measuring T cell population and/or PD-L1 expression in a tumor tissue sample from the subject; and
b. associating the T cell population and/or PD-L1 expression with the treatment response of the subject based on the relative abundance/percentage of the T cell population and/or PD-L1 expression status.

In another aspect, the disclosure relates to a method of treating cancer in a subject, the method comprising:
a. analyzing T cell population and/or PD-L1 expression in a tumor tissue sample from the subject; wherein the relative abundance/percentage of T cell population and/or PD-L1 positive status are indicative that the subject may benefit from the treatment; and
b. treating the subject in need of the treatment with a combination therapy comprising cabozantinib in combination with an immune checkpoint inhibitor or a cancer vaccine.

In another aspect, the disclosure relates to use of cabozantinib in combination with an immune checkpoint inhibitor or a cancer vaccine for the preparation of a medicament for the treatment of cancer in a subject, wherein the treatment comprises:
a. determining myeloid cell and T cell populations in a cancer sample from the subject, wherein the relative abundance/percentage of myeloid cell population and T cell population is indicative of the subject may benefit from the treatment; and
b. determining a therapeutically effective amount of cabozantinib for the treatment according to the relative abundance/percentage of myeloid cell population and T cell population.

In another aspect, the disclosure relates to a pharmaceutical composition comprising cabozantinib for use in combination with an immune checkpoint inhibitor or a cancer vaccine in treating cancer in a subject, wherein a cancer sample of the subject has a presence of myeloid CD68 cells and cytotoxic CD8 T-cells.

### BRIEF DESCRIPTION OF THE FIGURES

FIG. 1 tracks the association between TIL immune phenotype with lesion change in cancer patients treated with cabozantinib and an immune checkpoint inhibitor, atezolizumab.

FIG. 2A shows the association of PD-L1 with response and lesion change in ccRCC and nccRCC baseline tumor tissue. It shows that PD-L1 positive immune cells are significantly associated with overall response rate (ORR) response and lesion change from baseline in ccRCC cohort. Fractions of subjects (y-axis) that were PD-L1 positive or negative in CR/PR and SD/PD groups are indicated in the column graph. For the box plot, each dot represents a subject in the PD-L1 positive or negative group, plotted against the best lesion change from baseline.

FIG. 2B shows the association of CD8 with response and lesion change in ccRCC and nccRCC baseline tumor tissue. It shows that high frequency of CD8 T-cells cells is significantly associated with ORR response and lesion change from baseline in ccRCC cohort. Subjects that were CD8 high or CD8 low are compared with ORR in the column graph. For the box plot, subjects that were CD8 high or low are plotted against the best lesion change from baseline.

FIG. 2C shows the association of CD8 and CD68 with response and lesion change in ccRCC and nccRCC baseline tumor tissue. It shows that subjects who were CD8 T-cell high and CD68 high were significantly associated with ORR response and lesion change from baseline in ccRCC cohort. Fractions of subjects that were T-cell hot, Myeloid dominant, or immune cold, are indicated in the column graph by ORR. For the box plot, subjects that were T-cell hot, Myeloid dominant, or immune cold, are plotted against the best lesion change from baseline.

FIG. 2D shows the association of the CD8 T-cell density with ORR response. It shows that higher CD8 T-cell density in the tumor is significantly associated with ORR response. Subject CD8 T-cell density in the tumor or stromal margin region was compared in CR/PR to SD/PD.

FIG. 2E shows the correlation between the CD8 T-cell and progression free survival (PFS) in ccRCC hohort. It shows that high frequency of CD8 T-cells cells is associated with PFS survival in ccRCC cohort. The high CD8 T-cell group (dashed line) versus low (solid line) were plotted using a Kaplan-Meier curve. Hazard ratio (HR), confidence interval (parenthesis) and P-values (Cox proportional hazard model) in ccRCC cohort were labeled.

FIG. 3A shows the association of PD-L1 or CD8 with response and lesion change in ccRCC and nccRCC baseline tumor tissue. It shows that PD-L1 positive and/or high CD8 T-cells cells are significantly associated with ORR response and lesion change from baseline in ccRCC cohort. Fractions of subjects that were PD-L1 positive and/or CD8 high, e.g. Hot or Cold were compared by CR/PR and SD/PD response group in the column graph. For the box plot, each dot represents a subject in the Hot or Cold group, plotted against the best lesion change from baseline.

FIG. 3B shows the association of tumor CD68 and stroma CD68 with ORR response and lesion change in ccRCC and nccRCC baseline tumor tissue. It shows that tumor CD8 levels associate with lesion change from baseline in ccRCC cohort. Subject CD68 cell density in the tumor or stromal margin region was compared in CR/PR to SD/PD in the box plot. A pairwise plot compares best lesion change from baseline to CD8 or CD68 cell density in the tumor or stromal margin region colored by CR/PR or SD/PD.

FIG. 3C shows representative CD8 images that highlight regional differences in immune cell density. It shows the tumor and stromal margin regions for CD8 IHC staining.

### DETAILED DESCRIPTION

As used herein, the following definitions shall apply unless otherwise indicated.

For purposes of this invention, the chemical elements are identified in accordance with the Periodic Table of the Elements, CAS version, Handbook of Chemistry and Physics, 95th Ed. Additionally, general principles of organic chemistry are described in “Organic Chemistry,” 2nd Ed., Thomas Sorrell, University Science Books, Sausalito: 2006, and “March's Advanced Organic Chemistry,” 7th Ed., Ed.: Smith, M. B. and March, J., John Wiley & Sons, New York: 2013, the entire contents of which are hereby incorporated by reference.

“Patient” for the purposes of the present invention includes humans and any other animals, particularly mammals, and other organisms. Thus, the methods are applicable to both human therapy and veterinary applications. In a preferred embodiment, the patient is a mammal, and in a most preferred embodiment the patient is human. Examples of the preferred mammals include mice, rats, other rodents, rabbits, dogs, cats, swine, cattle, sheep, horses, and primates.

“Kinase-dependent diseases or conditions” refer to pathologic conditions that depend on the activity of one or more kinases. Kinases either directly or indirectly participate in the signal transduction pathways of a variety of cellular activities including proliferation, adhesion, migration, differentiation, and invasion. Diseases associated with kinase activities include tumor growth, the pathologic neovascularization that supports solid tumor growth, and associated with other diseases where excessive local vascularization is involved such as ocular diseases (diabetic retinopathy, age-related macular degeneration, and the like) and inflammation (psoriasis, rheumatoid arthritis, and the like).

“Therapeutically effective amount” is an amount of a crystalline form or crystalline salt form of the present invention that, when administered to a patient, ameliorates a symptom of the disease. The amount of a crystalline form or crystalline salt form of the present invention which constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit risk ratio.

As used herein, the phrase “pharmaceutically acceptable excipient” refers to a pharmaceutically-acceptable material, composition, or vehicle, such as a liquid or solid filler, diluent, solvent, or encapsulating material. Excipients are generally safe, non-toxic and neither biologically nor otherwise undesirable and include excipients that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of ‘Pharmaceutical Excipients, 6th ed.; Rowe et al, Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “concurrently” means at the same time. For example, if two treatment regimens for a single patient are being conducted concurrently, then they are being conducted at the same time. It will be understood that two treatment regimens happening at the same time, does not necessarily mean that actual delivery of two drugs happens at the same time, as each regimen may call for a different dosing schedule and/or different delivery modes.

“Cancer” refers to any physiological condition in mammals characterized by unregulated cell growth; in particular, cellular-proliferative disease states, including, but not limiting to: Cardiac: sarcoma (angiosarcoma, fibrosarcoma, rhabdomyosarcoma, liposarcoma), myxoma, rhabdomyoma, fibroma, lipoma and teratoma; Head and neck: squamous cell carcinomas of the head and neck, laryngeal and hypopharyngeal cancer, nasal cavity and paranasal sinus cancer, nasopharyngeal cancer, salivary gland cancer, oral and orppharyngeal cancer; Lung: bronchogenic carcinoma (squamous cell, undifferentiated small cell, undifferentiated large cell, adenocarcinoma, non-small cell lung cancer), alveolar (bronchiolar) carcinoma, alveolar sarcoma, alveolar soft part sarcoma, bronchial adenoma, sarcoma, lymphoma, chondromatous hamartoma, mesothelioma; Colon: colorectal cancer, adenocarcinoma, gastrointestinal stromal tumors, lymphoma, carcinoids, Turcot Syndrome; Gastrointestinal: gastric cancer, gastroesophageal junction adenocarcinoma, esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Breast: metastatic breast cancer, ductal carcinoma in situ, invasive ductal carcinoma, tubular carcinoma, medullary carcinoma, mucinous carcinoma, lobular carcinoma in situ, triple negative breast cancer; Genitourinary tract: kidney (adenocarcinoma, Wilm's tumor [nephroblastoma], lymphoma, leukemia, renal cell carcinoma, metastatic renal cell carcinoma, clear cell renal cell carcinoma), bladder and urethra (squamous cell carcinoma, transitional cell carcinoma, adenocarcinoma, urothelial carcinoma), prostate (adenocarcinoma, sarcoma, castrate resistant prostate cancer, bone metastases, bone metastases associated with castrate resistant prostate cancer,), testis (seminoma, teratoma, embryonal carcinoma, teratocarcinoma, choriocarcinoma, sarcoma, interstitial cell carcinoma, fibroma, fibroadenoma, adenomatoid tumors, lipoma), clear cell carcinoma, papillary carcinoma, penile cancer, penile squamous cell carcinoma; Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma, and giant cell tumors; Thyroid: medullary thyroid cancer, differentiated thyroid cancer, papillary thyroid cancer, follicular thyroid cancer, hurthle cell cancer, and anaplastic thyroid cancer; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma), NF1, neurofibromatosis, plexiform neurofibromas; Gynecological: uterus (endometrial cancer), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), myelofibrosis, polycythemia vera, essential thrombocythemia, Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions. In some embodiments, a compound or combination as disclosed herein can be used for the treatment of diseases including HIV, sickle cell disease, graft-versus-host disease, acute graft-versus-host disease, chronic graft-versus-host disease, and sickle cell anemia.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “enhance” refers to an increase or improvement in the function or activity of a protein or cell after administration or contacting with a combination described herein compared to the protein or cell prior to such administration or contact.

The term “administering” refers to the act of delivering a combination or composition described herein into a subject by such routes as oral, mucosal, topical, suppository, intravenous, parenteral, intraperitoneal, intramuscular, intralesional, intrathecal, intranasal or subcutaneous administration. Parenteral administration includes intravenous, intramuscular, intra-arteriole, intradermal, subcutaneous, intraperitoneal, intraventricular, and intracranial administration. Administration generally occurs after the onset of the disease, disorder, or condition, or its symptoms but, in certain instances, can occur before the onset of the disease, disorder, or condition, or its symptoms (e.g., administration for patients prone to such a disease, disorder, or condition).

The term “coadministration” refers to administration of two or more agents (e.g., a combination described herein and another active agent such as an anti-cancer agent described herein). The timing of coadministration depends in part of the combination and compositions administered and can include administration at the same time, just prior to, or just after the administration of one or more additional therapies, for example cancer therapies such as chemotherapy, hormonal therapy, radiotherapy, or immunotherapy. The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compounds described herein can be used in combination with one another, with other active agents known to be useful in treating cancer.

The term “anti-cancer agent” is used in accordance with its plain ordinary meaning and refers to a composition having anti-neoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

The term “chemotherapeutic” or “chemotherapeutic agent” is used in accordance with its plain ordinary meaning and refers to a chemical composition or compound having anti-neoplastic properties or the ability to inhibit the growth or proliferation of cells. “Chemotherapy” refers to a therapy or regimen that includes administration of a chemotherapeutic or anti-cancer agent described herein.

In general, the nomenclature used in this application is based on naming conventions adopted by the international union of pure and applied chemistry (IUPAC). Chemical structures shown herein were prepared using CHEMDRAW®. Any open valency appearing on a carbon, oxygen, or nitrogen atom in the structures herein indicates the presence of a hydrogen atom.

### Embodiments

### Methods

In one aspect, the disclosure relates to a method for predicting a treatment response of a subject having cancer to a therapy comprising cabozantinib in combination with an immune checkpoint inhibitor or a cancer vaccine, the method comprising:
a. measuring the myeloid cell and T cell populations in a tumor tissue sample from the subject; and
b. associating with the treatment response of the subject based on the relative abundance/percentage of myeloid cell population and T cell population.

In one embodiment of this aspect, the populations of myeloid cells and T cells are detected in the tumor tissue sample using a known method. In one embodiment, the myeloid cells are CD68 cells. In a further embodiment, the T cells are CD8 cells. In a further embodiment, the myeloid cells are CD68 cells and the T cells are CD8 cells. The relative abundance/percentage of CD68 and CD8 cells can indicate whether the subject may respond to the treatment. The abundance of myeloid CD68 cells and cytotoxic CD8 T-cells in the tumor indicates the responsiveness of the tumor to the therapy.

A variety of methods is available to measure myeloid cell and T cell populations in a tumor tissue sample according to step (a). These include qualitative or semi-quantitative scoring systems for Haemotoxylin and Eosin (H & E) stained specimens, which may vary according to tumor type. Alternatively, immunohistochemistry (IHC) can allow for the definition of the majority of immune cell subsets that can be further refined by combinations of markers, including, among others, CD8⁺ cytotoxic T cells, CD4⁺ T helper cells, FOXP3⁺ T_{regs}, B cells, macrophages and dendritic cells. Other cell types such as myeloid derived suppressor cells require multiple cell surface markers for definition and can be challenging to identify on serial IHC sections. Still alternatively, digital image analysis can provide accurate quantitation of immune cell infiltrates in IHC stained sections. Chromogenic IHC is another development for identifying cell populations. Chromogenic detection methods in IHC rely on enzymes that convert soluble substrates into insoluble, chromogenic products. Chromogenic detection is usually more sensitive than fluorescent detection because of the higher signal amplification involved in this method. Chromogenic stains are also more resistant to photobleaching that fluorochromes, resulting a longer listing signal.

Multiplexed fluorescent immunohistochemistry with multispectral imaging is another recent development that allows for in-situ identification of different immune cell subsets on the same section, thus giving quantitative information regarding the distribution and composition of immune infiltrate on formalin fixed, paraffin embedded (FFPE) tissue. Another recent approach utilizes NanoString® nCounter® fluorescent barcodes to identify bound antibodies. This allows for quantitation of multiple proteins in situ on an FFPE slide. FFPE tissue can also be used for matrix assisted laser desorption/ionization-imaging mass spectrometry (MALDI-IMS). MALDI-IMS is capable of identifying many of proteins in situ without requiring specific antibodies.

Alternatively, flow cytometry is a common approach to immune cell profiling. Flow cytometry allows for the characterization of immune cell subsets by multiple markers, quantitative data acquisition. Flow cytometry is a wide available technique that allows for the examination of small subpopulations. Flow cytometry requires fresh tissue, and does not provide information on the distribution or organization of the immune infiltrate or relationship to other microenvironmental structures.

Messenger RNA (mRNA) profiling of tumor tissue can detect “immune gene signatures”, using the level of expression of immune related genes to describe the composition and functional status of the immune infiltrate. No information is provided on the distribution of the infiltrate.

Thus, according to the method, a tumor tissue sample is harvested from a patient who has cancer. The patient is undergoing treatment with cabozantinib, along with an immune checkpoint inhibitor. The immune checkpoint inhibitor is but not limited to nivolumab, pembrolizumab, cemiplimab, durvalumab, atezolizumab or avelumab. In one embodiment, the immune checkpoint inhibitor is atezolizumab.

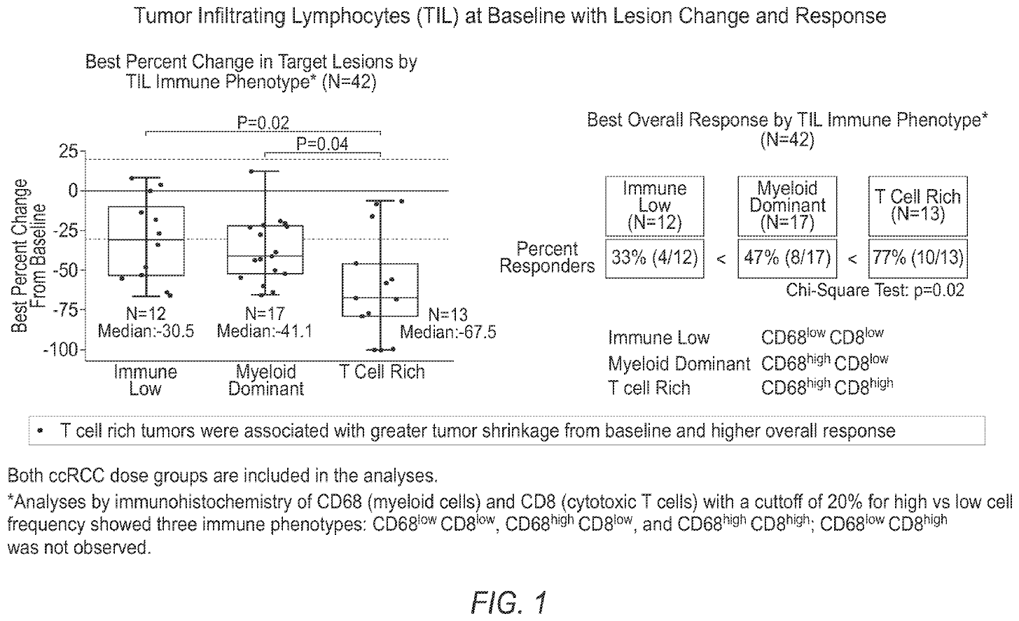

Using one or more of these methods, it is possible to determine the presence of myeloid (CD68) and cytotoxic T cells (CD8) in the tumor sample and to categorize the tumor samples by the amount of myeloid (CD68) and cytotoxic T cells (CD8) present. Referring to FIG. 1, the “Immune Low” phenotype is characterized by low levels of both CD68 and CD8 in tumor samples. The “Myeloid Dominant” phenotype is characterized by high levels of CD68 and low levels of CD8 in samples. The “T Cell Rich” phenotype is characterized by high levels of both CD68 and CD8 in tumor samples. The remaining phenotype is characterized by low levels of CD68 and high levels of CD8 in tumor samples. A high level of CD68 cells means 20% or greater of CD68 cell frequency or expression level according to the TIL counts measured by IHC. A high level of CD8 cells means 20% or greater of CD8 cell frequency or expression level according to the TIL counts measured by IHC. A low level of CD68 cells means less than 20% of CD68 cell frequency or expression level according to the TIL counts measured by IHC. A low level of CD8 cells means less than 20% of CD8 cell frequency or expression level according to the TIL counts measured by IHC.

The phenotype of the patient's tumor sample can be used to predict the response of the patient to an anti-tumor therapy. The “T Cell Rich” phenotype is associated with greater tumor shrinkage from baseline and overall response. A patient with a “T Cell Rich” tumor phenotype is administered one or more doses of cabozantinib and one or more doses of immune checkpoint inhibitor. The abundance of myeloid CD68 cells and cytotoxic CD8 T-cells in the tumor indicates the responsiveness of the tumor to treatment with cabozantinib and the checkpoint inhibitor therapy.

In another aspect, the disclosure provides a method for identifying a subject having cancer that has an increased benefit from a treatment comprising cabozantinib in combination with an immune checkpoint inhibitor or a cancer vaccine, the method comprising:

analyzing the myeloid cell and T cell populations in a tumor tissue sample from the subject, wherein the relative abundance/percentage of myeloid cell population and T cell population is indicative of the subject may benefit from the treatment.

In one embodiment of this aspect, the presence of myeloid cells and T cells are detected in the tumor tissue sample using a known method. In one embodiment, the myeloid cells are CD68 cells. In a further embodiment, the T cells are CD8 cells. In a further embodiment, the myeloid cells are CD68 cells and the T cells are CD8 cells. The size of the populations of CD68 and CD8 cells can indicate whether the subject may experience a greater benefit to the treatment. The presence of myeloid CD68 cells and cytotoxic CD8 T-cells in the tumor indicates the responsiveness of the tumor to treatment with cabozantinib and the checkpoint inhibitor therapy.

In an embodiment, the level of CD68 cells measured from tumors is at least 20% according to the TIL counts measured by IHC. In a further embodiment, the level of CD8 cells measured from tumors is at least 20% according to the TIL counts measured by IHC. In a further embodiment, the levels of CD68 and CD8 measured from tumors are each least 20% measured by IHC. In a further embodiment, the level of CD68 cells or CD8 cells measured from tumors is below 20% according to the TIL counts measured by IHC. In a further embodiment, the levels of CD68 cells and CD8 cells measured from tumors are each below 20% according to the TIL counts measured by IHC.

In these and other embodiments, tumors with levels of CD68 and CD8 that are each independently at least 20 percent (i.e., “T Cell Rich” phenotype tumors) are more likely to respond to cabozantinib/checkpoint inhibitor (i.e., atezolizumab) treatment, as evidenced by the reduction of lesion size relative to baseline. Tumors where one or both of CD8 or CD68 levels fall below 20 percent as measured by IHC do not respond as well to cabozantinib/checkpoint inhibitor (i.e., atezolizumab) treatment, as evidenced by the less tumor shrinkage compared to the “T Cell Rich” phenotype tumors. Thus, a presence of at least 20% of both CD68 and CD8 cells in the tumor (i.e., “T Cell Rich” phenotype tumors) indicates that the subject is mostly likely benefit from the treatment. The likelihood that the subject may benefit from the treatment is less if the CD68 cells are at least 20% and the CD8 cells are less than 20% (i.e., “Myeloid Dominant” phenotype tumors). If both CD68 and CD8 cells are less than 20% (i.e., “Immune Low” phenotype tumors), the likelihood the subject may benefit from the treatment is lower compared to the other two types.

Referring again to FIG. 1, the “T Cell Rich” phenotype is characterized by relatively large populations and thus relatively large abundances of both CD68 and CD8 in tumor samples, and the “T Cell Rich” phenotype is associated with greater tumor shrinkage from baseline and overall response. A patient with a “T Cell Rich” tumor phenotype who is administered one or more doses of cabozantinib and one or more doses of immune checkpoint inhibitor would be predicted to benefit from the treatment.

In one embodiment, the method is directed to identifying a subject having cancer that exhibit an increased benefit from a treatment comprising cabozantinib in combination with an immune checkpoint inhibitor or a cancer vaccine, the method comprising:

analyzing the myeloid cell and T cell populations in a tumor tissue sample from the subject being treated with comprising cabozantinib in combination with an immune checkpoint inhibitor or a cancer vaccine, wherein the relative abundance/percentage of myeloid cell population and T cell population is indicative of the subject may benefit from the treatment.

In a further aspect, the disclosure relates to a method of treating cancer in a subject, the method comprising:
analyzing the myeloid cell and T cell populations in a tumor tissue sample from the subject; wherein the relative abundance/percentage of myeloid cell population and T cell population is indicative of the subject may benefit from the treatment; and
treating the subject in need of the treatment with a combination therapy comprising cabozantinib in combination with an immune checkpoint inhibitor or a cancer vaccine.

In one embodiment of this aspect, the populations of myeloid cells and T cells are detected in the tumor tissue sample using a known method. In one embodiment, the myeloid cells are CD68 cells. In a further embodiment, the T cells are CD8 cells. In a further embodiment, the myeloid cells are CD68 cells and the T cells are CD8 cells. The size of the populations of CD68 and CD8 cells can indicate whether the subject may experience a greater benefit to the treatment. The presence of myeloid CD68 cells and cytotoxic CD8 T-cells in the tumor indicates the responsiveness of the tumor to treatment with cabozantinib and the checkpoint inhibitor therapy.

In an embodiment, the level of CD68 cells measured from tumors is at least 20% according to the TIL counts measured by IHC. In a further embodiment, the level of CD8 cells measured from tumors is at least 20% according to the TIL counts measured by IHC. In a further embodiment, the levels of CD68 and CD8 measured from tumors are each least 20% measured by IHC. In a further embodiment, the level of CD68 cells or CD8 cells measured from tumors is below 20% according to the TIL counts measured by IHC. In a further embodiment, the levels of CD68 cells and CD8 cells measured from tumors are each below 20% according to the TIL counts measured by IHC.

In these and other embodiments, tumors with levels of CD68 and CD8 that are each independently at least 20 percent (i.e., “T Cell Rich” phenotype tumors) are more likely to respond to cabozantinib/checkpoint inhibitor (i.e., atezolizumab) treatment, as evidenced by the lesion relative to baseline. Tumors where one or both of CD8 or CD68 levels fall below 20 percent as measured by IHC do not respond as well to cabozantinib/checkpoint inhibitor (i.e., atezolizumab) treatment, as evidenced by the less tumor shrinkage compared to the “T Cell Rich” phenotype tumors. Thus, a presence of at least 20% of both CD68 and CD8 cells in the tumor (i.e., “T Cell Rich” phenotype tumors) indicates that the subject is mostly likely benefit from the treatment. The likelihood that the subject may benefit from the treatment is less if the CD68 cells are at least 20% and the CD8 cells are less than 20% (i.e., “Myeloid Dominant” phenotype tumors). If both CD68 and CD8 cells are less than 20% (i.e., “Immune Low” phenotype tumors), the likelihood the subject may benefit from the treatment is lower compared to the other types.

Referring again to FIG. 1, the “T Cell Rich” phenotype is characterized by relatively large populations and thus relatively large abundances of both CD68 and CD8 in tumor samples, and the “T Cell Rich” phenotype is associated with greater tumor shrinkage from baseline and overall response.

In one embodiment, the method is directed to treating cancer in a patient having a “T Cell Rich” phenotype tumor, comprising administering one or more doses of cabozantinib and one or more doses of an immune checkpoint inhibitor or cancer treatment of the patient.

In another aspect, the disclosure relates to a method of treating a subject having a solid tumor with cabozantinib in combination with immune checkpoint inhibitor or an anti-cancer vaccine, comprising:
a. determining whether the solid tumor comprises myeloid CD68 cells and cytotoxic CD8 T-cells; and
b if the tumor comprises myeloid CD68 cells and cytotoxic CD8 T-cells, administering one or more doses of cabozantinib and one or more doses of immune checkpoint inhibitor therapy to the subject, wherein the presence of myeloid CD68 cells and cytotoxic CD8 T-cells in the tumor indicates the responsiveness of the tumor to treatment with cabozantinib and checkpoint inhibitor therapy.

In one embodiment of this aspect, the presence of myeloid cells and T cells are detected in the tumor tissue sample using a known method. In one embodiment, the myeloid cells are CD68 cells. In a further embodiment, the T cells are CD8 cells. In a further embodiment, the myeloid cells are CD68 cells and the T cells are CD8 cells. The size of the populations of CD68 and CD8 cells can indicate whether the subject may experience a greater benefit to the treatment. The presence of myeloid CD68 cells and cytotoxic CD8 T-cells in the tumor indicates the responsiveness of the tumor to treatment with the checkpoint inhibitor therapy.

In an embodiment, the level of CD68 cells measured from tumors is at least 20% according to the TIL counts measured by IHC. In a further embodiment, the level of CD8 cells measured from tumors is at least 20% according to the TIL counts measured by IHC. In a further embodiment, the levels of CD68 and CD8 measured from tumors are each least 20% measured by IHC. In a further embodiment, the level of CD68 cells or CD8 cells measured from tumors is below 20% according to the TIL counts measured by IHC. In a further embodiment, the levels of CD68 cells and CD8 cells measured from tumors are each below 20% according to the TIL counts measured by IHC.

In these and other embodiments, tumors with levels of CD68 and CD8 that are each independently at least 20 percent (i.e., “T Cell Rich” phenotype tumors) are more likely to respond to cabozantinib/checkpoint inhibitor (i.e., atezolizumab) treatment, as evidenced by the lesion relative to baseline. Tumors where one or both of CD8 or CD68 levels fall below 20 percent as measured by IHC do not respond as well to cabozantinib/checkpoint inhibitor (i.e., atezolizumab) treatment, as evidenced by the less tumor shrinkage compared to the “T Cell Rich” phenotype tumors. Thus, a presence of at least 20% of both CD68 and CD8 cells in the tumor (i.e., “T Cell Rich” phenotype tumors) indicates that the subject is mostly likely benefit from the treatment. The likelihood that the subject may benefit from the treatment is less if the CD68 cells are at least 20% and the CD8 cells are less than 20% (i.e., “Myeloid Dominant” phenotype tumors). If both CD68 and CD8 cells are less than 20% (i.e., “Immune Low” phenotype tumors), the likelihood the subject may benefit from the treatment is lower compared to the other types.

Referring again to FIG. 1, the “T Cell Rich” phenotype is characterized by relatively large populations and thus relatively large abundances of both CD68 and CD8 in tumor samples, and the “T Cell Rich” phenotype is associated with greater tumor shrinkage from baseline and overall response.

In one embodiment, the method is directed to treating cancer in a patient having a “T Cell Rich” phenotype tumor, comprising:
a. determining whether the solid tumor is a “T Cell Rich” phenotype tumor; and
b if the tumor is a “T Cell Rich” phenotype tumor, administering one or more doses of cabozantinib and one or more doses of immune checkpoint inhibitor therapy or cancer vaccine to the subject, wherein the “T Cell Rich” phenotype tumor indicates the responsiveness of the tumor to treatment with the checkpoint inhibitor therapy.

In another aspect, the disclosure relates to a method of determining whether to treat a patient having a solid tumor with cabozantinib in combination with immune checkpoint inhibitor or an anti-cancer vaccine, the method comprising:
determining the populations of myeloid CD68 cells and cytotoxic CD8 T-cells in the solid tumor; and
determining to treat the patient with a therapeutically effective amount of cabozantinib and a therapeutically effective amount of immune checkpoint inhibitor or an anti-cancer vaccine according to the population of the myeloid CD68 cells and cytotoxic CD8 T-cells.

In an embodiment, the level of CD68 cells measured from tumors is at least 20% according to the TIL counts measured by IHC. In a further embodiment, the level of CD8 cells measured from tumors is at least 20% according to the TIL counts measured by IHC. In a further embodiment, the levels of CD68 and CD8 measured from tumors are each least 20% measured by IHC. In a further embodiment, the level of CD68 cells or CD8 cells measured from tumors is below 20% according to the TIL counts measured by IHC. In a further embodiment, the levels of CD68 cells and CD8 cells measured from tumors are each below 20% according to the TIL counts measured by IHC.

In these and other embodiments, tumors with levels of CD68 and CD8 that are each independently at least 20 percent (i.e., “T Cell Rich” phenotype tumors) are more likely to respond to cabozantinib/checkpoint inhibitor (i.e., atezolizumab) treatment, as evidenced by the magnitude of lesion relative to baseline. Tumors where one or both of CD8 or CD68 levels fall below 20 percent as measured by IHC do not respond as well to cabozantinib/checkpoint inhibitor (i.e., atezolizumab) treatment, as evidenced by the less tumor shrinkage compared to the “T Cell Rich” phenotype tumors.

Thus, in a further embodiment, the method comprises determining to treat the patient if a presence of 20% or greater of both CD68 and CD8 cells is detected. In a further embodiment, the method comprises determining to treat the patient if a presence of 20% or greater of CD68 cells and less than 20% CD8 cells is detected. In a further embodiment, the method comprises determining to treat the patient if both CD68 and CD8 cells are less than 20%.

In a further embodiment, the method comprises determining to treat the patient if the tumor has high levels of CD68 and CD8 cells; high level of CD68 and low level of CD8 cells; or low levels of CD68 and CD8 cells. In one embodiment, the method comprises determining to treat the patient if the tumor has high levels of both myeloid CD68 cells and cytotoxic CD8 T-cells. In one embodiment, the method comprises determining to treat the patient if the tumor is a “T Cell Rich” phenotype tumor.

In another aspect, the disclosure relates to a method for predicting a treatment response of a subject having cancer to a therapy comprising cabozantinib in combination with an immune checkpoint inhibitor or a cancer vaccine, the method comprising:
a. measuring T cell population in a tumor tissue sample from the subject; and
b. associating the T cell population with the treatment response of the subject based on the relative abundance/percentage of the T cell population.

In another aspect, the disclosure relates to a method for predicting a treatment response of a subject having cancer to a therapy comprising cabozantinib in combination with an immune checkpoint inhibitor or a cancer vaccine, the method comprising:
a. measuring PD-L1 expression in a tumor tissue sample from the subject; and
b. associating the PD-L1 expression with the treatment response of the subject based on PD-L1 expression status.

In another aspect, the disclosure relates to a method for predicting a treatment response of a subject having cancer to a therapy comprising cabozantinib in combination with an immune checkpoint inhibitor or a cancer vaccine, the method comprising:
a. measuring T cell population and PD-L1 expression in a tumor tissue sample from the subject; and
b. associating the T cell population and PD-L1 expression with the treatment response of the subject based on the relative abundance/percentage of the T cell population and PD-L1 expression status.

In another aspect, the disclosure relates to a method for identifying a subject having cancer as a candidate for treatment with a therapy comprising cabozantinib in combination with an immune checkpoint inhibitor or a cancer vaccine, the method comprising:

analyzing T cell population and/or PD-L1 expression in a tumor tissue sample from the subject, wherein the the relative abundance/percentage of the T cell population and/or PD-L1 positive status indicates that the subject may benefit from the treatment with cabozantinib in combination with the immune checkpoint inhibitor or cancer vaccine.

In another aspect, the disclosure relates to a method of treating cancer in a subject, the method comprising:
a. analyzing T cell population in a tumor tissue sample from the subject; wherein the relative abundance/percentage of T cell population is indicative that the subject may benefit from the treatment; and
b. treating the subject in need of the treatment with a combination therapy comprising cabozantinib in combination with an immune checkpoint inhibitor or a cancer vaccine.

In another aspect, the disclosure relates to a method of treating cancer in a subject, the method comprising:
a. analyzing PD-L1 expression in a tumor tissue sample from the subject; wherein a PD-L1 positive status is indicative that the subject may benefit from the treatment; and
b. treating the subject in need of the treatment with a combination therapy comprising cabozantinib in combination with an immune checkpoint inhibitor or a cancer vaccine.

In another aspect, the disclosure relates to a method of treating cancer in a subject, the method comprising:
a. analyzing T cell population and PD-L1 expression in a tumor tissue sample from the subject; wherein the relative abundance/percentage of T cell population and PD-L1 positive status are indicative that the subject may benefit from the treatment; and
b. treating the subject in need of the treatment with a combination therapy comprising cabozantinib in combination with an immune checkpoint inhibitor or a cancer vaccine.

In some embodiments, the T cell is CD8 cells and the tumor tissue sample has high levels of CD8 cells.

In some embodiments, the tumor tissue sample has PD-L1 positive immune cells.

In another aspect, the disclosure relates to a method of treating a subject having a solid tumor with cabozantinib in combination with an immune checkpoint inhibitor, the method comprising:
a. determining CD8 T cell level in a tumor tissue sample from the subject; and
b. administering one or more doses of cabozantinib and one or more doses of the immune checkpoint inhibitor to the subject if the tumor comprises CD8 T cells, wherein the presence of CD8 T cells indicates the responsiveness of the tumor to treatment with the checkpoint inhibitor therapy.

In another aspect, the disclosure relates to a method of treating a subject having a solid tumor with cabozantinib in combination with an immune checkpoint inhibitor, the method comprising:
a. determining PD-L1 expression in a tumor tissue sample from the subject; and
b. administering one or more doses of cabozantinib and one or more doses of the immune checkpoint inhibitor to the subject if the tumor comprises PD-L1 positive immune cells, wherein the presence of PD-L1 positive immune cells indicates the responsiveness of the tumor to treatment with the checkpoint inhibitor therapy.

In another aspect, the disclosure relates to a method of treating a subject having a solid tumor with cabozantinib in combination with an immune checkpoint inhibitor, the method comprising:
a. determining CD8 T cell level and PD-L1 expression in a tumor tissue sample from the subject; and
b. administering one or more doses of cabozantinib and one or more doses of the immune checkpoint inhibitor to the subject if the tumor comprises CD8 T cells and PD-L1 positive immune cells, wherein the presence of CD8 T cells and PD-L1 positive immune cells indicates the responsiveness of the tumor to treatment with the checkpoint inhibitor therapy.

In some embodiments, the method comprises administering one or more doses of cabozantinib and one or more doses of immune checkpoint inhibitor therapy to the subject if the tumor comprises high level of CD8 T-cells.

In some embodiments, the method comprises administering one or more doses of cabozantinib and one or more doses of immune checkpoint inhibitor therapy to the subject if the tumor comprises PD-L1 positive immune cells.

In another aspect, the disclosure relates to a method of treating clear cell renal cell carcinoma, the method comprising:
a. determining CD8 T cell level and/or PD-L1 expression in a tumor tissue sample from the subject; and
b. administering one or more doses of cabozantinib and one or more doses of atezolizumab to the subject if the tumor comprises CD8 T cells and/or PD-L1 positive immune cells, wherein the presence of CD8 T cells and/or PD-L1 positive immune cells indicates the responsiveness of the tumor to treatment with the checkpoint inhibitor therapy.

In another aspect, the disclosure relates to use of cabozantinib in combination with an immune checkpoint inhibitor or a cancer vaccine for the preparation of a medicament for the treatment of cancer in a subject, wherein the treatment comprises:
a. determining myeloid cell and T cell populations in a cancer sample from the subject, wherein the relative abundance/percentage of myeloid cell population and T cell population is indicative of the subject may benefit from the treatment; and
b. determining a therapeutically effective amount of cabozantinib for the treatment according to the relative abundance/percentage of myeloid cell population and T cell population.

In another aspect, the disclosure relates to a pharmaceutical composition comprising cabozantinib for use in combination with an immune checkpoint inhibitor or a cancer vaccine in treating cancer in a subject, wherein a cancer sample of the subject has a presence of myeloid CD68 cells and cytotoxic CD8 T-cells.

In another aspect, the disclosure relates to use of cabozantinib in combination with an immune checkpoint inhibitor or a cancer vaccine for the preparation of a medicament for the treatment of cancer in a subject, wherein the treatment comprises:
a. determining T cell populations and/or PD-L1 expression in a cancer sample from the subject, wherein the relative abundance/percentage of T cell population and/or PD-L1 expression status is indicative that the subject may benefit from the treatment; and
b. determining a therapeutically effective amount of cabozantinib for the treatment according to the relative abundance/percentage of T cell population and/or PD-L1 expression status.

In another aspect, the disclosure relates to a pharmaceutical composition comprising cabozantinib for use in combination with an immune checkpoint inhibitor or a cancer vaccine in treating cancer in a subject, wherein a cancer sample of the subject has a presence of cytotoxic CD8 T-cells.

In another aspect, the disclosure relates to a pharmaceutical composition comprising cabozantinib for use in combination with an immune checkpoint inhibitor or a cancer vaccine in treating cancer in a subject, wherein a cancer sample of the subject has PD-L1 positive immune cells.

In one embodiment, the use is direct to treating cancer in the subject, wherein the subject has “T Cell Rich” phenotype tumor, wherein the “T Cell Rich” phenotype tumor indicates the responsiveness of the tumor to treatment with cabozantinib and the checkpoint inhibitor therapy.

### Cabozantinib

The embodiments disclosed herein employ cabozantinib, which is an oral inhibitor of tyrosine kinases including MET, VEGF receptors, and AXL. Cabozantinib has the structure depicted below.

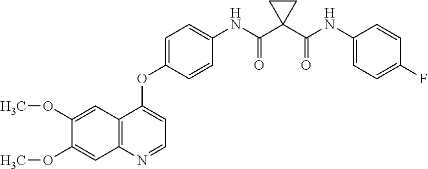 [SMILES: COc1cc2nccc(Oc3ccc(NC(=O)C4(C(=O)Nc5ccc(F)cc5)CC4)cc3)c2cc1OC]

Cabozantinib has been approved in capsule (COMETRIQ®) and tablet (CABOMETYX®) formulations for the treatment of, among other indications, medullary thyroid cancer, renal cell carcinoma, and hepatocellular carcinoma. Cabozantinib is administered as the (S)-malate salt. Cabozantinib (5)-malate is described chemically as N-(4-(6,7-dimethoxyquinolin-4-yloxy)phenyl)-N′-(4-fluorophenyl)cyclopropane-1,1-dicarboxamide, (2S)-hydroxybutanedioate. The molecular formula is C₂₈H₂₄FN₃O₅·C₄H₆O₅, and the molecular weight is 635.6 Daltons as malate salt. The chemical structure of cabozantinib (S)-malate salt is depicted below.

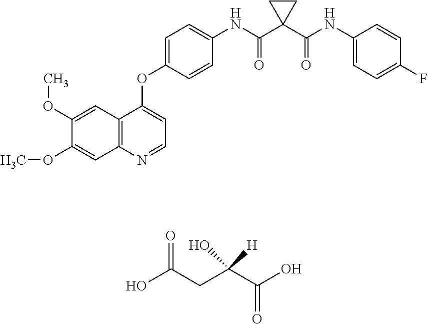 [SMILES: COc1cc2nccc(Oc3ccc(NC(=O)C4(C(=O)Nc5ccc(F)cc5)CC4)cc3)c2cc1OC ; O=C(O)C[C@H](O)C(=O)O]

Cabozantinib as a capsule formulation (COMETRIQ®) has been approved for the treatment of medullary thyroid cancer.

Cabozantinib is administered as a tablet formulation containing cabozantinib containing cabozantinib as the S-malate. The amount of cabozantinib contained in each tablet is from 20 to 80 mg (free base equivalent). In one embodiment, the tablet contains 20, 25, 30, 35, 40, 45, 50, 55, or 60 mg of cabozantinib. The dose of cabozantinib administered is typically from 20 to 60 mg.

In an embodiment, the cabozantinib is administered as CABOMETYX®, which is a tablet formulation of the (S)-malate salt of cabozantinib.

In a further embodiment is adminstered as a tablet formulation comprising:
30-32 percent by weight of cabozantinib S-malate salt wherein cabozantinib is:

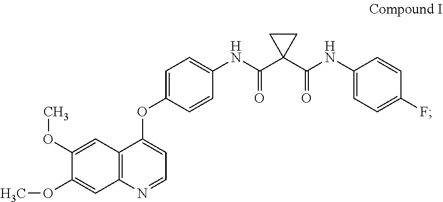 [SMILES: COc1cc2nccc(Oc3ccc(NC(=O)C4(C(=O)Nc5ccc(F)cc5)CC4)cc3)c2cc1OC]

38-40 percent by weight of microcrystalline cellulose;
18-22 percent by weight of lactose;
2-4 percent by weight of hydroxypropyl cellulose;
4-8 percent by weight of croscarmellose sodium;
0.2-0.6 percent by weight of colloidal silicon dioxide;
0.5-1 percent by weight of magnesium stearate; and
3.9-4.1 percent by weight of a film coating material comprising hypromellose, titanium dioxide, triacetin, and iron oxide yellow.

### Immune Checkpoint Inhibitor

The embodiments disclosed herein employ cabozantinib in combination with an immune checkpoint inhibitor.

Checkpoint inhibitors can include treatments, molecules, agents, and/or methods that regulate immune checkpoints at the transcriptional level, e.g., using the RNA-interference pathway co-suppression, and/or post-transcriptional gene silencing (PTGS) (e.g., microRNAs, miRNA; silencing-RNA, small-interfering-RNA, or short-interfering-RNA (siRNA).

Transcriptional regulation of checkpoint molecules has been shown to involve mir-16, which has been shown to target the 3′UTR of the checkpoint mRNAs CD80, CD274 (PD-L1) and CD40 (Leibowitz et al., Post-transcriptional regulation of immune checkpoint genes by mir-16 in melanoma, Annals of Oncology (2017) 28; v428-v448). Mir-33a has also been shown to be involved in regulating the expression of PD-1 in cases of lung adenocarcinoma (Boldini et al., Role of microRNA-33a in regulating the expression of PD-1 in lung adenocarcinoma, Cancer Cell Int. 2017; 17: 105, the disclosure of which is incorporated herein by reference in its entirety).

T-cell-specific aptamer-siRNA chimeras have been suggested as a highly specific method of inhibiting molecules in the immune checkpoint pathway (Hossain et al., The aptamer-siRNA conjugates: reprogramming T cells for cancer therapy, Ther. Deliv. 2015 January; 6(1): 1-4, the disclosure of which is incorporated herein by reference in its entirety).

Alternatively, members of the immune checkpoint pathway can be inhibited using treatments that affect associated pathways, e.g., metabolism. For example, oversupplying the glycolytic intermediate pyruvate in mitochondria from CAD macrophages promoted expression of PD-L1 via induction of the bone morphogenetic protein 4/phosphorylated SMAD1/5/IFN regulatory factor 1 (BMP4/p-SMAD1/5/IRF1) signaling pathway. Accordingly, implementing treatments that modulate the metabolic pathway can result in subsequent modulation of the immunoinhibitory PD-1/PD-L1 checkpoint pathway (Watanabe et al., Pyruvate controls the checkpoint inhibitor PD-L1 and suppresses T cell immunity, J Clin Invest. 2017 Jun. 30; 127(7): 2725-2738).

Checkpoint immunity can be regulated via oncolytic viruses that selectively replicate within tumor cells and induce acute immune responses in the tumor-micro-environment, i.e., by acting as genetic vectors that carry specific agents (e.g., antibodies, miRNA, siRNA, and the like) to cancer cells and effecting their oncolysis and secretion of cytokines and chemokines to synergize with immune checkpoint inhibition (Shi et al., Cancer Immunotherapy: A Focus on the Regulation of Immune Checkpoints, Int J Mol Sci. 2018 May; 19(5): 1389). Currently, there are clinical trials underway that utilize the following viruses as checkpoint inhibitors: poliovirus, measles virus, adenoviruses, poxviruses, herpes simplex virus (HSV), coxsackieviruses, reovirus, Newcastle disease virus (NDV), T-VEC (a herpes virus encoded with GM-CSF (granulocyte-macrophage colony stimulating factor)), and H101 (Shi et al., supra).

Checkpoint inhibitors can operate at the translational level of checkpoint immunity. The translation of mRNA into protein represents a key event in the regulation of gene expression, thus inhibition of immune checkpoint translation is a method in which the immune checkpoint pathway can be inhibited.

Inhibition of the immune checkpoint pathway can occur at any stage of the immune checkpoint translational process. For example, drugs, molecules, agents, treatments, and/or methods can inhibit the initiation process (whereby the 40S ribosomal subunit is recruited to the 5′ end of the mRNA and scans the 5′UTR of the mRNA toward its 3′ end. Inhibition can occur by targeting the anticodon of the initiator methionyl-transfer RNA (tRNA) (Met-tRNAi), its base-pairing with the start codon, or the recruitment of the 60S subunit to begin elongation and sequential addition of amino acids in the translation of immune-checkpoint-specific genes. Alternatively, a checkpoint inhibitor can inhibit checkpoints at the translational level by preventing the formation of the ternary complex (TC), i.e., eukaryotic initiation factor (eIF)2 (or one or more of its α, β, and γ subunits); GTP; and Met-tRNAi.

Checkpoint inhibition can occur via destabilization of eIF2a by precluding its phosphorylation via protein kinase R (PKR), PERK, GCN2, or HRI, or by precluding TCs from associating with the 40S ribosome and/or other initiation factors, thus preventing the preinitiation complex (PIC) from forming; inhibiting the eIF4F complex and/or its cap-binding protein eIF4E, the scaffolding protein eIF4G, or eIF4A helicase. Methods discussing the translational control of cancer are discussed in Truitt et al., New frontiers in translational control of the cancer genome, Nat Rev Cancer. 2016 Apr. 26; 16(5): 288-304, the disclosure of which is incorporated herein by reference in its entirety.

Checkpoint inhibitors can also include treatments, molecules, agents, and/or methods that regulate immune checkpoints at the cellular and/or protein level, e.g., by inhibiting an immune checkpoint receptor. Inhibition of checkpoints can occur via the use of antibodies, antibody fragments, antigen-binding fragments, small-molecules, and/or other drugs, agents, treatments, and/or methods.

Immune checkpoints refer to inhibitory pathways in the immune system that are responsible for maintaining self-tolerance and modulating the degree of immune system response to minimize peripheral tissue damage. However, tumor cells can also activate immune system checkpoints to decrease the effectiveness of immune response (‘block’ the immune response) against tumor tissues. In contrast to the majority of anti-cancer agents, checkpoint inhibitors do not target tumor cells directly, but rather target lymphocyte receptors or their ligands in order to enhance the endogenous antitumor activity of the immune system. (Pardoll, 2012, Nature Reviews Cancer 12:252-264).

In some embodiments, the immunotherapeutic agent is a modulator of PD-1 activity, a modulator of PD-L1 activity, a modulator of PD-L2 activity, a modulator of CTLA-4 activity, a modulator of CD28 activity, a modulator of CD80 activity, a modulator of CD86 activity, a modulator of 4-1BB activity, an modulator of OX40 activity, a modulator of KIR activity, a modulator of Tim-3 activity, a modulator of LAG3 activity, a modulator of CD27 activity, a modulator of CD40 activity, a modulator of GITR activity, a modulator of TIGIT activity, a modulator of CD20 activity, a modulator of CD96 activity, a modulator of IDO1 activity, a cytokine, a chemokine, an interferon, an interleukin, a lymphokine, a member of the tumor necrosis factor (TNF) family, or an immunostimulatory oligonucleotide. In some embodiments, the immune checkpoint modulator, i.e. is an inhibitor or antagonist, or is an activator or agonist, for example, a CD28 modulator, a 4-1BB modulator, an OX40 modulator, a CD27 modulator, a CD80 modulator, a CD86 modulator, a CD40 modulator, or a GITR modulator, a Lag-3 modulator, a 41BB modulator, a LIGHT modulator, a CD40 modulator, a GITR modulator, a TGF-beta modulator, a TIM-3 modulator, a SIRP-alpha modulator, a TIGIT modulator, a VSIG8 modulator, a BTLA modulator, a SIGLEC7 modulator, a SIGLEC9 modulator, a ICOS modulator, a B7H3 modulator, a B7H4 modulator, a FAS modulator, and/or a BTNL2 modulator. In some embodiments, the immunotherapeutic agent is an immune checkpoint modulator as described above (e.g., an immune checkpoint modulator antibody, which can be in the form of a monoclonal antibody, a bispecific antibody comprising one or more immune checkpoint antigen binding moieties, a trispecific antibody, or an immune cell-engaging multivalent antibody/fusion protein/construct known in the art).

In some embodiments, the immunotherapeutic agent is an agent that inhibits the activity of PD-1. In some embodiments, the immunotherapeutic agent is an agent that inhibits the activity of PD-L1 and/or PD-L2. In some embodiments, the immunotherapeutic agent is an agent that inhibits the activity of CTLA-4. In some embodiments, the immunotherapeutic agent is an agent that inhibits the activity of CD80 and/or CD86. In some embodiments, the immunotherapeutic agent is an agent that inhibits the activity of TIGIT. In some embodiments, the immunotherapeutic agent is an agent that inhibits the activity of MR. In some embodiments, the immunotherapeutic agent is an agent that enhances or stimulates the activity of activating immune checkpoint receptors.

PD-1 (also known as Programmed Death 1, CD279, PDCD1) is a cell surface receptor with a critical role in regulating the balance between stimulatory and inhibitory signals in the immune system and maintaining peripheral tolerance (Ishida, Y et al. 1992 EMBO J. 11 3887; Kier, Mary E et al. 2008 Annu Rev Immunol 26 677-704; Okazaki, Taku et al. 2007 International Immunology 19 813-824). PD-1 is an inhibitory member of the immunoglobulin super-family with homology to CD28. The structure of PD-1 is a monomeric type 1 transmembrane protein, consisting of one immunoglobulin variable-like extracellular domain and a cytoplasmic domain containing an immunoreceptor tyrosine-based inhibitory motif (ITIM) and an immunoreceptor tyrosine-based switch motif (ITSM). Expression of PD-1 is inducible on T cells, B cells, natural killer (NK) cells and monocytes, for example upon lymphocyte activation via T cell receptor (TCR) or B cell receptor (BCR) signalling (Kier, Mary E et al. 2008 Annu Rev Immunol 26 677-704; Agata, Y et al 1996 Int Immunol 8 765-72). PD-1 is a receptor for the ligands CD80, CD86, PD-L1 (B7-H1, CD274) and PD-L2 (B7-DC, CD273), which are cell surface expressed members of the B7 family (Freeman, Gordon et al. 2000 J Exp Med 192 1027; Latchman, Y et al. 2001 Nat Immunol 2: 261). Upon ligand engagement, PD-1 recruits phosphatases such as SHP-1 and SHP-2 to its intracellular tyrosine motifs which subsequently dephosphorylate effector molecules activated by TCR or BCR signalling (Chemnitz, J et al. 2004 J Immunol 173: 945-954; Riley, James L 2009 Immunological Reviews 229: 114-125) In this way, PD-1 transduces inhibitory signals into T and B cells only when it is engaged simultaneously with the TCR or BCR.

PD-1 has been demonstrated to down-regulate effector T cell responses via both cell-intrinsic and cell-extrinsic functional mechanisms. Inhibitory signaling through PD-1 induces a state of unresponsiveness in T cells, resulting in the cells being unable to clonally expand or produce optimal levels of effector cytokines. PD-1 may also induce apoptosis in T cells via its ability to inhibit survival signals from co-stimulation, which leads to reduced expression of key anti-apoptotic molecules such as Bcl-XL (Kier, Mary E et al. 2008 Annu Rev Immunol 26: 677-704). In addition to these direct effects, recent publications have implicated PD-1 as being involved in the suppression of effector cells by promoting the induction and maintenance of regulatory T cells (TREG). For example, PD-L1 expressed on dendritic cells was shown to act in synergy with TGF-β to promote the induction of CD4+ FoxP3+TREG with enhanced suppressor function (Francisco, Loise M et al. 2009 J Exp Med 206: 3015-3029).

TIM-3 (also known as T-cell immunoglobulin and mucin-domain containing-3, TIM-3, Hepatitis A virus cellular receptor 2, HAVCR2, HAVcr-2, KIM-3, TIMD-3, TIMD3, Tim-3, and CD366) is a ˜33.4-kDa single-pass type I membrane protein involved in immune responses (Sanchez-Fueyo et al., Tim-3 inhibits T helper type 1-mediated auto- and alloimmune responses and promotes immunological tolerance, Nat. Immunol. 4: 1093-1101(2003)). TIM-3 is selectively expressed on Th1-cells, and phagocytic cells (e.g., macrophages and dendritic cells). The use of siRNA or a blocking antibody to reduce the expression of human TIM-3 resulted in increased secretion of interferon γ (IFN-γ) from CD4 positive T-cells, implicating the inhibitory role of TIM-3 in human T cells. Analysis of clinical samples from autoimmune disease patients showed no expression of TIM-3 in CD4 positive cells. In particular, expression level of TIM-3 is lower and secretion of IFN-γ is higher in T cell clones derived from the cerebrospinal fluid of patients with multiple sclerosis than those in clones derived from normal healthy persons (Koguchi K et al., J Exp Med. 203: 1413-8. (2006)).

TIM-3 is the receptor for the ligand Galectin-9, which is a member of galectin family, molecules ubiquitously expressed on a variety of cell types and which binds β-galactoside; Phospatidyl serine (PtdSer) (DeKryff et al., T cell/transmembrane, Ig, and mucin-3 allelic variants differentially recognize phosphatidylserine and mediate phagocytosis of apoptotic cells, J Immunol. 2010 Feb. 15; 184(4): 1918-30); High Mobility Group Protein 1 (also known as HMGB1, HMG1, HMG3, SBP-1, HMG-1, and high mobility group box 1) Chiba et al., Tumor-infiltrating DCs suppress nucleic acid-mediated innate immune responses through interactions between the receptor TIM-3 and the alarmin HMGB1, Nat Immunol. 2012 September; 13(9): 832-42); and Carcinoembryonic Antigen Related Cell Adhesion Molecule 1 (also known as CEACAM1, BGP, BGP1, BGPI, carcinoembryonic antigen related cell adhesion molecule 1) (Huang et al., CEACAM1 regulates TIM-3-mediated tolerance and exhaustion, Nature. 2015 Jan. 15; 517(7534): 386-90).

BTLA (also known as B- and T-lymphocyte attenuator, BTLA1, CD272, and B and T lymphocyte associated) is a ˜27.3-kDa single-pass type I membrane protein involved in lymphocyte inhibition during immune response. BTLA is constitutively expressed in both B and T cells. BTLA interacts with HVEM (herpes virus-entry mediator), a member of the tumor-necrosis factor receptor (TNFR) family (Gonzalez et al., Proc. Natl. Acad. Sci. USA, 2005, 102: 1116-21). The interaction of BTLA, which belongs to the CD28 family of the immunoglobulin superfamily, and HVEM, a costimulatory tumor-necrosis factor (TNF) receptor (TNFR), is unique in that it defines a cross talk between these two families of receptors. BTLA contains a membrane proximal immunoreceptor tyrosine-based inhibitory motif (ITIM) and membrane distal immunoreceptor tyrosine-based switch motif (ITSM). Disruption of either the ITIM or ITSM abrogated the ability of BTLA to recruit either SHP1 or SHP2, suggesting that BTLA recruits SHP1 and SHP2 in a manner distinct from PD-1 and both tyrosine motifs are required to block T cell activation. The BTLA cytoplasmic tail also contains a third conserved tyrosine-containing motif within the cytoplasmic domain, similar in sequence to a Grb-2 recruitment site (YXN). Also, a phosphorylated peptide containing this BTLA N-terminal tyrosine motif can interact with GRB2 and the p85 subunit of PI3K in vitro, although the functional effects of this interaction remain unexplored in vivo (Gavrieli et al., Bioochem. Biophysi Res Commun, 2003, 312, 1236-43). BTLA is the receptor for the ligands PTPN6/SHP-1; PTPN11/SHP-2; TNFRSF14/HVEM; and B7H4.

VISTA (also known as V-domain Ig suppressor of T cell activation VSIR, B7-H5, B7H5, GI24, PP2135, SISP1, DD1 alpha, VISTA, C10orf54, chromosome 10 open reading frame 54, PD-1H, and V-set immunoregulatory receptor) is a ˜33.9-kDa single-pass type I membrane protein involved in T-cell inhibitory response, embryonic stem cells differentiation via BMP4 signaling inhibition, and MMP14-mediated MMP2 activation (Yoon et al., Control of signaling-mediated clearance of apoptotic cells by the tumor suppressor p53, Science. 2015 Jul. 31; 349(6247): 1261669). VISTA interacts with the ligand VSIG-3 (Wang et al., VSIG-3 as a ligand of VISTA inhibits human T-cell function, Immunology. 2019 January; 156(1): 74-85)

LAG-3 (also known as Lymphocyte-activation gene 3, LAG3, CD223, and lymphocyte activating 3) is a ˜57.4-kDa single-pass type I membrane protein involved in lymphocyte activation that also binds to HLA class-II antigens. LAG-3 is a member of the immunoglobulin supergene family, and is expressed on activated T cells (Huard et al., 1994, Immunogenetics 39: 213), NK cells (Triebel et al., 1990, J. Exp. Med. 171: 1393-1405), regulatory T cells (Huang et al., 2004, Immunity 21: 503-513; Camisaschi et al., 2010, J Immunol. 184: 6545-6551; Gagliani et al., 2013, Nat Med 19: 739-746), and plasmacytoid dendritic cells (DCs) (Workman et al., 2009, J Immunol 182: 1885-1891). LAG-3 is a membrane protein encoded by a gene located on chromosome 12, and is structurally and genetically related to CD4. Similar to CD4, LAG-3 can interact with MHC class II molecules on the cell surface (Baixeras et al., 1992, J. Exp. Med. 176: 327-337; Huard et al., 1996, Eur. J. Immunol. 26: 1180-1186). It has been suggested that the direct binding of LAG-3 to MHC class II plays a role in down-regulating antigen-dependent stimulation of CD4+ T lymphocytes (Huard et al., 1994, Eur. J. Immunol. 24: 3216-3221) and LAG-3 blockade has also been shown to reinvigorate CD8+ lymphocytes in both tumor or self-antigen (Gross et al., 2007, J Clin Invest. 117: 3383-3392) and viral models (Blackburn et al., 2009, Nat. Immunol. 10: 29-37). Further, the intra-cytoplasmic region of LAG-3 can interact with LAP (LAG-3-associated protein), which is a signal transduction molecule involved in the downregulation of the CD3/TCR activation pathway (Iouzalen et al., 2001, Eur. J. Immunol. 31: 2885-2891). Moreover, CD4+CD25+ regulatory T cells (Treg) have been shown to express LAG-3 upon activation, which contributes to the suppressor activity of Treg cells (Huang, C. et al., 2004, Immunity 21: 503-513). LAG-3 can also negatively regulate T cell homeostasis by Treg cells in both T cell-dependent and independent mechanisms (Workman, C. J. and Vignali, D. A., 2005, J. Immunol. 174: 688-695).

LAG-3 has been shown to interact with MHC class II molecules (Huard et al., CD4/major histocompatibility complex class II interaction analyzed with CD4- and lymphocyte activation gene-3 (LAG-3)-Ig fusion proteins, Eur J Immunol. 1995 September; 25(9): 2718-21).

Additionally, several kinases are known to be checkpoint inhibitors. For example, CHEK-1, CHEK-2, and A2aR.

CHEK-1 (also known as CHK 1 kinase, CHK1, and checkpoint kinase 1) is a ˜54.4-kDa serine/threonine-protein kinase that is involved with checkpoint-mediated cell cycle arrest, and the activation of DNA repair in response to the DNA damage and/or unreplicated DNA.

CHEK-2 (also known as CHK2 kinase, CDS1, CHK2, HuCds1, LFS2, PP1425, RAD53, hCds1, and checkpoint kinase 2) is a ˜60.9-kDa. serine/threonine-protein kinase involved in checkpoint-mediated cell cycle arrest, DNA-repair activation, and double-strand break-mediated apoptosis.

A2aR (also known as adenosine A2A receptor, ADORA2A, adenosine A2a receptor, A2aR, ADORA2, and RDC8) is a ˜44.7-kDa multi-pass membrane receptor for adenosine and other ligands.

In some embodiments, illustrative immunotherapeutic agents can include one or more antibody modulators that target PD-1, PD-L1, PD-L2, CEACAM (e.g., CEACAM-1, -3 and/or -5), CTLA-4, TIM-3, LAG-3, VISTA, BTLA, TIGIT, LAIR1, CD160, 2B4, TGF beta, OX40, 41BB, LIGHT, CD40, GITR, TGF-beta, TIM-3, SIRP-alpha, VSIG8, BTLA, SIGLEC7, SIGLEC9, ICOS, B7H3, B7H4, FAS, and/or BTNL2 among others known in the art. In some embodiments, the immunotherapeutic agent is an agent that increases natural killer (NK) cell activity. In some embodiments, the immunotherapeutic agent is an agent that inhibits suppression of an immune response. In some embodiments, the immunotherapeutic agent is an agent that inhibits suppressor cells or suppressor cell activity. In some embodiments, the immunotherapeutic agent is an agent or therapy that inhibits Treg activity. In some embodiments, the immunotherapeutic agent is an agent that inhibits the activity of inhibitory immune checkpoint receptors.

In some embodiments, cabozantinib and an immunotherapeutic agent, wherein the immunotherapeutic agent includes a T cell modulator chosen from an agonist or an activator of a costimulatory molecule. In one embodiment, the agonist of the costimulatory molecule is chosen from an agonist (e.g., an agonistic antibody or antigen-binding fragment thereof, or a soluble fusion) of GITR, OX40, SLAM (e.g., SLAMF7), HVEM, LIGHT, CD2, CD27, CD28, CDS, ICAM-1, LFA-1 (CD11a/CD18), ICOS (CD278), 4-1BB (CD137), CD30, CD40, BAFFR, CD7, NKG2C, NKp80, CD160, B7-H3, or CD83 ligand. In other embodiments, the effector cell combination includes a bispecific T cell engager (e.g., a bispecific antibody molecule that binds to CD3 and a tumor antigen (e.g., EGFR, PSCA, PSMA, EpCAM, HER2 among others).

In some embodiments, the immunotherapeutic agent is a modulator of PD-1 activity, a modulator of PD-L1 activity, a modulator of PD-L2 activity, a modulator of CTLA-4 activity, a modulator of CD28 activity, a modulator of CD80 activity, a modulator of CD86 activity, a modulator of 4-1BB activity, an modulator of OX40 activity, a modulator of KIR activity, a modulator of Tim-3 activity, a modulator of LAG3 activity, a modulator of CD27 activity, a modulator of CD40 activity, a modulator of GITR activity, a modulator of TIGIT activity, a modulator of CD20 activity, a modulator of CD96 activity, a modulator of IDO1 activity, a modulator of SIRP-alpha activity, a modulator of TIGIT activity, a modulator of VSIG8 activity, a modulator of BTLA activity, a modulator of SIGLEC7 activity, a modulator of SIGLEC9 activity, a modulator of ICOS activity, a modulator of B7H3 activity, a modulator of B7H4 activity, a modulator of FAS activity, a modulator of BTNL2 activity, a cytokine, a chemokine, an interferon, an interleukin, a lymphokine, a member of the tumor necrosis factor (TNF) family, or an immunostimulatory oligonucleotide.

In some embodiments, the immunotherapeutic agent is an immune checkpoint modulator (e.g., an immune checkpoint inhibitor e.g. an inhibitor of PD-1 activity, a modulator of PD-L1 activity, a modulator of PD-L2 activity, a modulator of CTLA-4, or a CD40 agonist (e.g., an anti-CD40 antibody molecule), (xi) an OX40 agonist (e.g., an anti-OX40 antibody molecule), or (xii) a CD27 agonist (e.g., an anti-CD27 antibody molecule). In one embodiment, the immunotherapeutic agent is an inhibitor of: PD-1, PD-L1, PD-L2, CTLA-4, TIM-3, LAG-3, CEACAM (e.g., CEACAM-1, -3 and/or -5), VISTA, BTLA, TIGIT, LAIR1, CD160, 2B4 and/or TGF beta, Galectin 9, CD69, Galectin-1, CD113, GPR56, CD48, GARP, PD1H, LAIR1, TIM-1, and TIM-4. In one embodiment, the inhibitor of an immune checkpoint molecule inhibits PD-1, PD-L1, LAG-3, TIM-3, CEACAM (e.g., CEACAM-1, -3 and/or -5), CTLA-4, or any combination thereof.

In one embodiment, the immunotherapeutic agent is an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, the immunotherapeutic agent used in the combinations disclosed herein (e.g., in combination with cabozantinib) is an activator or agonist of a costimulatory molecule. In one embodiment, the agonist of the costimulatory molecule is chosen from an agonist (e.g., an agonistic antibody or antigen-binding fragment thereof, or a soluble fusion) of CD2, CD28, CDS, ICAM-1, LFA-1 (CD11a/CD18), ICOS (CD278), 4-1BB (CD137), GITR, CD30, BAFFR, HVEM, CD7, LIGHT, NKG2C, SLAMF7, NKp80, CD160, B7-H3, or CD83 ligand.

Inhibition of an inhibitory molecule can be performed at the DNA, RNA or protein level. In embodiments, an inhibitory nucleic acid (e.g., a dsRNA, siRNA or shRNA), can be used to inhibit expression of an inhibitory molecule. In other embodiments, the inhibitor of an inhibitory signal is, a polypeptide e.g., a soluble ligand (e.g., PD-1-Ig or CTLA-4 Ig), or an antibody or antigen-binding fragment thereof, for example, a monoclonal antibody, a bispecific antibody comprising one or more immune checkpoint antigen binding moieties, a trispecific antibody, or an immune cell-engaging multivalent antibody/fusion protein/construct known in the art that binds to the inhibitory molecule; e.g., an antibody or fragment thereof (also referred to herein as “an antibody molecule”) that binds to PD-1, PD-L1, PD-L2, CTLA-4, TIM-3, LAG-3, CEACAM (e.g., CEACAM-1, -3 and/or -5), VISTA, BTLA, TIGIT, LAIR′, CD160, 2B4 and/or TGF beta, Galectin 9, CD69, Galectin-1, CD113, GPR56, CD48, GARP, PD1H, LAIR1, TIM-1, TIM-4, or a combination thereof.

In some embodiments, where the combination comprises cabozantinib and an immunotherapeutic agent, wherein the immunotherapeutic agent is a monoclonal antibody or a bispecific antibody. For example, the monoclonal or bispecific antibody may specifically bind a member of the c-Met pathway and/or an immune checkpoint modulator (e.g., the bispecific antibody binds to both a hepatocyte growth factor receptor (HGFR) and an immune checkpoint modulator described herein, such as an antibody that binds PD-1, PD-L1, PD-L2, or CTLA-4, LAG-3, OX40, 41BB, LIGHT, CD40, GITR, TGF-beta, TIM-3, SIRP-alpha, TIGIT, VSIG8, BTLA, SIGLEC7, SIGLEC9, ICOS, B7H3, B7H4, FAS, BTNL2 or CD27). In particular embodiments, the bispecific antibody specifically binds a human HGFR protein and one of PD-1, PD-L1, and CTLA-4.

In some of the embodiments of the methods described herein, the immunotherapeutic agent is a PD-1 antagonist, a PD-L1 antagonist, a PD-L2 antagonist, a CTLA-4 antagonist, a CD80 antagonist, a CD86 antagonist, a MR antagonist, a Tim-3 antagonist, a LAG3 antagonist, a TIGIT antagonist, a CD20 antagonist, a CD96 antagonist, or an IDO1 antagonist.

In some embodiments, the PD-1 antagonist is an antibody that specifically binds PD-1. In some embodiments, the antibody that binds PD-1 is pembrolizumab (KEYTRUDA®, MK-3475; Merck), pidilizumab (CT-011; Curetech Ltd.), nivolumab (OPDIVO®, BMS-936558, MDX-1106; Bristol Myer Squibb), MEDI0680 (AMP-514; AstraZenenca/MedImmune), REGN2810 (Regeneron Pharmaceuticals), BGB-A317 (BeiGene Ltd.), PDR-001 (Novartis), or STI-A1110 (Sorrento Therapeutics). In some embodiments, the antibody that binds PD-1 is described in PCT Publication WO 2014/179664, for example, an antibody identified as APE2058, APE1922, APE1923, APE1924, APE 1950, or APE1963 (Anaptysbio), or an antibody containing the CDR regions of any of these antibodies. In other embodiments, the PD-1 antagonist is a fusion protein that includes the extracellular domain of PD-L1 or PD-L2, for example, AMP-224 (AstraZeneca/MedImmune). In other embodiments, the PD-1 antagonist is a peptide inhibitor, for example, AUNP-12 (Aurigene).

In some embodiments, the PD-L1 antagonist is an antibody that specifically binds PD-L1. In some embodiments, the antibody that binds PD-L1 is atezolizumab (RG7446, MPDL3280A; Genentech), MEDI4736 (AstraZeneca/MedImmune), BMS-936559 (MDX-1105; Bristol Myers Squibb), avelumab (MSB0010718C; Merck KGaA), KD033 (Kadmon), the antibody portion of KD033, or STI-A1014 (Sorrento Therapeutics). In some embodiments, the antibody that binds PD-L1 is described in PCT Publication WO 2014/055897, for example, Ab-14, Ab-16, Ab-30, Ab-31, Ab-42, Ab-50, Ab-52, or Ab-55, or an antibody that contains the CDR regions of any of these antibodies, the disclosure of which is incorporated herein by reference in its entirety.

In some embodiments, the CTLA-4 antagonist is an antibody that specifically binds CTLA-4. In some embodiments, the antibody that binds CTLA-4 is ipilimumab (YERVOY®; Bristol Myer Squibb) or tremelimumab (CP-675, 206; Pfizer). In some embodiments, the CTLA-4 antagonist a CTLA-4 fusion protein or soluble CTLA-4 receptor, for example, KARR-102 (Kahr Medical Ltd.).

In some embodiments, the LAG3 antagonist is an antibody that specifically binds LAG3. In some embodiments, the antibody that binds LAG3 is IMP701 (Prima BioMed), IMP731 (Prima BioMed/GlaxoSmithKline), BMS-986016 (Bristol Myer Squibb), LAG525 (Novartis), and GSK2831781 (GlaxoSmithKline). In some embodiments, the LAG3 antagonist includes a soluble LAG3 receptor, for example, IMP321 (Prima BioMed).

In some embodiments, the MR antagonist is an antibody that specifically binds MR. In some embodiments, the antibody that binds MR is lirilumab (Bristol Myer Squibb/Innate Pharma).

In some embodiments, the immunotherapeutic agent is a cytokine, for example, a chemokine, an interferon, an interleukin, lymphokine, or a member of the tumor necrosis factor family. In some embodiments, the cytokine is IL-2, IL15, or interferon-gamma.

In some embodiments of any of the above aspects or those described elsewhere herein, the cancer is selected from the group consisting of lung cancer (e.g., a non-small cell lung cancer (NSCLC)), a kidney cancer (e.g., a kidney urothelial carcinoma), a bladder cancer (e.g., a bladder urothelial (transitional cell) carcinoma), a breast cancer, a colorectal cancer (e.g., a colon adenocarcinoma), an ovarian cancer, a pancreatic cancer, a gastric carcinoma, an esophageal cancer, a mesothelioma, a melanoma (e.g., a skin melanoma), a head and neck cancer (e.g., a head and neck squamous cell carcinoma (HNSCC)), a thyroid cancer, a sarcoma (e.g., a soft-tissue sarcoma, a fibrosarcoma, a myxosarcoma, a liposarcoma, an osteogenic sarcoma, an osteosarcoma, a chondrosarcoma, an angiosarcoma, an endotheliosarcoma, a lymphangiosarcoma, a lymphangioendotheliosarcoma, a leiomyosarcoma, or a rhabdomyosarcoma), a prostate cancer, a glioblastoma, a cervical cancer, a thymic carcinoma, a leukemia (e.g., an acute lymphocytic leukemia (ALL), an acute myelocytic leukemia (AML), a chronic myelocytic leukemia (CML), a chronic eosinophilic leukemia, or a chronic lymphocytic leukemia (CLL)), a lymphoma (e.g., a Hodgkin lymphoma or a non-Hodgkin lymphoma (NHL)), a myeloma (e.g., a multiple myeloma (MM)), a mycoses fungoides, a merkel cell cancer, a hematologic malignancy, a cancer of hematological tissues, a B cell cancer, a bronchus cancer, a stomach cancer, a brain or central nervous system cancer, a peripheral nervous system cancer, a uterine or endometrial cancer, a cancer of the oral cavity or pharynx, a liver cancer, a testicular cancer, a biliary tract cancer, a small bowel or appendix cancer, a salivary gland cancer, an adrenal gland cancer, adrenal cortex carcinoma, an adenocarcinoma, an inflammatory myofibroblastic tumor, a gastrointestinal stromal tumor (GIST), a colon cancer, a myelodysplastic syndrome (MDS), a myeloproliferative disorder (MPD), a polycythemia Vera, a chordoma, a synovioma, an Ewing's tumor, a squamous cell carcinoma, a basal cell carcinoma, an adenocarcinoma, a sweat gland carcinoma, a sebaceous gland carcinoma, a papillary carcinoma, a papillary adenocarcinoma, a medullary carcinoma, a bronchogenic carcinoma, a renal cell carcinoma, a clear cell renal cell carcinoma (ccRCC), a hepatoma, a bile duct carcinoma, a choriocarcinoma, a seminoma, an embryonal carcinoma, a Wilms' tumor, a bladder carcinoma, an epithelial carcinoma, a glioma, anaplastic astrocytoma, an astrocytoma, a medulloblastoma, a craniopharyngioma, an ependymoma, a pinealoma, a hemangioblastoma, an acoustic neuroma, an oligodendroglioma, a meningioma, a neuroblastoma, a retinoblastoma, a follicular lymphoma, a diffuse large B-cell lymphoma, a mantle cell lymphoma, a hepatocellular carcinoma, a thyroid cancer, a small cell cancer, an essential thrombocythemia, an agnogenic myeloid metaplasia, a hypereosinophilic syndrome, a systemic mastocytosis, a familiar hypereosinophilia, a neuroendocrine cancer, or a carcinoid tumor. In another embodiment of any of the above aspects or those described elsewhere herein, the cancer is selected from the group consisting of medullary thyroid cancer, renal cell carcinoma, clear cell renal cell carcinoma (ccRCC), and hepatocellular carcinoma. In another embodiment, the cancer is ccRCC.

In some embodiments of any of the above aspects or those described elsewhere herein, the subject's cancer or tumor does not respond to immune checkpoint inhibition (e.g., to any immune checkpoint inhibitor described herein, such as a PD-1 antagonist or PD-L1 antagonist) or the subject's cancer or tumor has progressed following an initial response to immune checkpoint inhibition (e.g., to any immune checkpoint inhibitor described herein, such as a PD-1 antagonist or PD-L1 antagonist).

In various embodiments, the immunotherapeutic agent can comprise an antibody or an antigen binding fragment thereof. Within this definition, immune checkpoint inhibitors include bispecific antibodies and immune cell-engaging multivalent antibody/fusion protein/constructs known in the art. In some embodiments, immunotherapeutic agents which comprise bispecific antibodies may include bispecific antibodies that are bivalent and bind either the same epitope of the immune checkpoint molecule, two different epitopes of the same immune checkpoint molecule or different epitopes of two different immune checkpoints.

Persons of ordinary skill in the art can implement several bispecific antibody formats known in the field to target one or more of CTLA4, PD1, PD-L1 TIM-3, LAG-3, various B-7 ligands, B7H3, B7H4, CHK 1 and CHK2 kinases, BTLA, A2aR, OX40, 41BB, LIGHT, CD40, GITR, TGF-beta, SIRP-alpha, TIGIT, VSIG8, SIGLEC7, SIGLEC9, ICOS, FAS, BTNL2 and other for use in the combination described herein.

In various embodiments, the immunotherapeutic agent can include am immune cell-engaging multivalent antibody/fusion protein/construct.

In an embodiment of the disclosure, the checkpoint inhibitor, in combination with cabozantinib, is used to reduce or inhibit metastasis of a primary tumor or cancer to other sites, or the formation or establishment of metastatic tumors or cancers at other sites distal from the primary tumor or cancer thereby inhibiting or reducing tumor or cancer relapse or tumor or cancer progression.

In a further embodiment of the disclosure, provided herein is a combination therapy for treating cancer, which comprises cabozantinib and a checkpoint inhibitor with the potential to elicit potent and durable immune responses with enhanced therapeutic benefit and more manageable toxicity.

In a further embodiment of the disclosure, provided herein is a combination therapy for treating cancer, which comprises cabozantinib and an immune checkpoint inhibitor. In an embodiment of the disclosure provided herein is a method for treating cancer and/or preventing the establishment of metastases by employing cabozantinib, which acts synergistically with a checkpoint inhibitor.

In further embodiments, the disclosure provides methods for one or more of the following: 1) reducing or inhibiting growth, proliferation, mobility or invasiveness of tumor or cancer cells that potentially or do develop metastases, 2) reducing or inhibiting formation or establishment of metastases arising from a primary tumor or cancer to one or more other sites, locations or regions distinct from the primary tumor or cancer; 3) reducing or inhibiting growth or proliferation of a metastasis at one or more other sites, locations or regions distinct from the primary tumor or cancer after a metastasis has formed or has been established, 4) reducing or inhibiting formation or establishment of additional metastasis after the metastasis has been formed or established, 5) prolonged overall survival, 6) prolonged progression free survival, or 7) disease stabilization. The methods include administering to a subject in need thereof cabozantinib, in combination with a check point inhibitor as described herein.

In an embodiment of the disclosure, administration of cabozantinib in combination with the immunotherapeutic agent, provides a detectable or measurable improvement in a condition of a given subject, such as alleviating or ameliorating one or more adverse (physical) symptoms or consequences associated with the presence of a cell proliferative or cellular hyperproliferative disorder, neoplasia, tumor or cancer, or metastasis, i.e., a therapeutic benefit or a beneficial effect.

A therapeutic benefit or beneficial effect is any objective or subjective, transient, temporary, or long-term improvement in the condition or pathology, or a reduction in onset, severity, duration or frequency of adverse symptom associated with or caused by cell proliferation or a cellular hyperproliferative disorder such as a neoplasia, tumor or cancer, or metastasis. It may lead to improved survival. A satisfactory clinical endpoint of a treatment method in accordance with the disclosure is achieved, for example, when there is an incremental or a partial reduction in severity, duration or frequency of one or more associated pathologies, adverse symptoms or complications, or inhibition or reversal of one or more of the physiological, biochemical or cellular manifestations or characteristics of cell proliferation or a cellular hyperproliferative disorder such as a neoplasia, tumor or cancer, or metastasis. A therapeutic benefit or improvement therefore may be, but is not limited to destruction of target proliferating cells (e.g., neoplasia, tumor or cancer, or metastasis) or ablation of one or more, most or all pathologies, adverse symptoms or complications associated with or caused by cell proliferation or the cellular hyperproliferative disorder such as a neoplasia, tumor or cancer, or metastasis. However, a therapeutic benefit or improvement need not be a cure or complete destruction of all target proliferating cells (e.g., neoplasia, tumor or cancer, or metastasis) or ablation of all pathologies, adverse symptoms or complications associated with or caused by cell proliferation or the cellular hyperproliferative disorder such as a neoplasia, tumor or cancer, or metastasis. For example, partial destruction of a tumor or cancer cell mass, or a stabilization of the tumor or cancer mass, size or cell numbers by inhibiting progression or worsening of the tumor or cancer, can reduce mortality and prolong lifespan even if only for a few days, weeks or months, even though a portion or the bulk of the tumor or cancer mass, size or cells remain.

Specific non-limiting examples of therapeutic benefit include a reduction in neoplasia, tumor or cancer, or metastasis volume (size or cell mass) or numbers of cells; inhibiting or preventing an increase in neoplasia, tumor or cancer volume (e.g., stabilizing); slowing or inhibiting neoplasia, tumor or cancer progression, worsening or metastasis; or inhibiting neoplasia, tumor or cancer proliferation, growth or metastasis.

In an embodiment of the disclosure, administration of the immunotherapeutic agent, in combination therapy with cabozantinib, provides a detectable or measurable improvement or overall response according to the irRC (as derived from time-point response assessments and based on tumor burden), including one of more of the following: (i) irCR—complete disappearance of all lesions, whether measurable or not, and no new lesions (confirmation by a repeat, consecutive assessment no less than 4 weeks from the date first documented), (ii) irPR—decrease in tumor burden ≥50% relative to baseline (confirmed by a consecutive assessment at least 4 weeks after first documentation).

Optionally, any method described herein may not take effect immediately. For example, treatment may be followed by an increase in the neoplasia, tumor or cancer cell numbers or mass, but over time eventual stabilization or reduction in tumor cell mass, size or numbers of cells in a given subject may subsequently occur.

Additional adverse symptoms and complications associated with neoplasia, tumor, cancer and metastasis that can be inhibited, reduced, decreased, delayed or prevented include, for example, nausea, lack of appetite, lethargy, pain and discomfort. Thus, a partial or complete decrease or reduction in the severity, duration or frequency of adverse symptom or complication associated with or caused by a cellular hyperproliferative disorder, an improvement in the subject's quality of life and/or well-being, such as increased energy, appetite, psychological well-being, are all particular non-limiting examples of therapeutic benefit.

A therapeutic benefit or improvement therefore can also include a subjective improvement in the quality of life of a treated subject. In an additional embodiment, a method prolongs or extends lifespan (survival) of the subject. In a further embodiment, a method improves the quality of life of the subject.

In one embodiment, administration of the immunotherapeutic agent, in combination therapy with cabozantinib, results in a clinically relevant improvement in one or more markers of disease status and progression selected from one or more of the following: (i) overall survival, (ii) progression-free survival, (iii) overall response rate, (iv) reduction in metastatic disease, (v) circulating levels of tumor antigens such as carbohydrate antigen 19.9 (CA19.9) and carcinembryonic antigen (CEA) or others depending on tumor, (vii) nutritional status (weight, appetite, serum albumin), (viii) pain control or analgesic use, and (ix) CRP/albumin ratio.

Treatment with cabozantinib in combination with an immunotherapeutic agent gives rise to more complex immunity including not only the development of innate immunity and type-1 immunity, but also immunoregulation which more efficiently restores appropriate immune functions.

In various exemplary methods, a checkpoint inhibitor antibody (monoclonal or polyclonal, bispecific, trispecific, or an immune cell-engaging multivalent antibody/fusion protein/construct) directed to a checkpoint molecule of interest (e.g., PD-1) may be sequenced and the polynucleotide sequence may then be cloned into a vector for expression or propagation. The sequence encoding the antibody or antigen-binding fragment thereof of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., 2008, J. Immunol. Methods 329: 112; U.S. Pat. No. 7,314,622.

Formulations of the an immunotherapeutic agent, for example an immune checkpoint modulator antibody used in accordance with the present disclosure can be prepared for storage by mixing an antibody having the desired degree of purity with optional pharmaceutically acceptable excipients or stabilizers as amply described and illustrated in Remington's Pharmaceutical Sciences 21st Ed., (Lippincott, Williams and Wilkins Philadelphia, PA, 2006), in the form of lyophilized formulations or aqueous solutions and/or suspensions. Acceptable excipients, buffers or stabilizers are nontoxic to recipients at the dosages and concentrations employed, and include suitable aqueous and/or non-aqueous excipients that may be employed in the pharmaceutical compositions of the disclosure, for example, water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants, buffers such as phosphate, citrate, and other organic acids. Antioxidants may be included, for example, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like; preservatives (such as octade-cyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues). Other exemplary pharmaceutically acceptable excipients may include polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In another exemplary embodiment, one or more immunotherapeutic agents, or an antigen-binding fragment thereof is formulated for intravenous or subcutaneous administration as a sterile aqueous solution containing 1-75 mg/mL, or more preferably, about 5-60 mg/mL, or yet more preferably, about 10-50 mg/mL, or even more preferably, about 10-40 mg/mL of antibody, with sodium acetate, polysorbate 80, and sodium chloride at a pH ranging from about 5 to 6. Preferably, the intravenous or subcutaneous formulation is a sterile aqueous solution containing 5, 10, 15, 20, 25, 30, 35, 40, 45, or 50 mg/mL of the immunotherapeutic agent, for example, an immune checkpoint inhibitor antibody or an antigen-binding fragment thereof, with 20 mM sodium acetate, 0.2 mg/mL polysorbate 80, and 140 mM sodium chloride at pH 5.5. Further, a solution comprising a checkpoint inhibitor antibody or an antigen-binding fragment thereof, can comprise, among many other compounds, histidine, mannitol, sucrose, trehalose, glycine, poly(ethylene)glycol, EDTA, methionine, and any combination thereof, and many other compounds known in the relevant art.

In one embodiment, part of the immunotherapeutic agent is administered by an intravenous bolus and the rest by infusion of the immunotherapeutic agent formulation. For example, from about 0.001 to about 200 mg/kg, for example, from about 0.001 mg/kg to about 100 mg/kg, or from about 0.001 mg/kg to about 50 mg/kg, or from about 0.001 mg/kg to about 10 mg/kg intravenous injection of the immunotherapeutic agent, or antigen-binding fragment thereof, may be given as a bolus, and the rest of the antibody dose may be administered by intravenous injection. A predetermined dose of the immunotherapeutic agent, or antigen-binding fragment thereof, may be administered, for example, over a period of an hour to two hours to five hours.

In a further embodiment, part of the immunotherapeutic agent is administered by a subcutaneous injection and/or infusion in the form of a bolus and the rest by infusion of the immunotherapeutic agent formulation. In some exemplary doses, the immunotherapeutic agent formulation can be administered subcutaneously in a dose ranging from about 0.001 to about 200 mg/kg, for example, from about 0.001 mg/kg to about 100 mg/kg, or from about 0.001 mg/kg to about 50 mg/kg, or from about 0.001 mg/kg to about 10 mg/kg intravenous injection of the immunotherapeutic agent, or antigen-binding fragment thereof. In some embodiments the dose may be given as a bolus, and the rest of the immunotherapeutic agent dose may be administered by subcutaneous or intravenous injection. A predetermined dose of the immunotherapeutic agent, or antigen-binding fragment thereof, may be administered, for example, over a period of an hour to two hours to five hours.

### Anti-Cancer Vaccine

The embodiments disclosed herein employ cabozantinib in combination with an anti-cancer vaccine. In various embodiments, an illustrative immunotherapeutic agent comprises one or more cancer vaccines, for use in combination with cabozantinib. The tumor-associated antigen component of the vaccine may be manufactured by any of a variety of well-known techniques. For individual protein components, the antigenic protein is isolated from tumor tissue or a tumor-cell line by standard chromatographic means such as high-pressure liquid chromatography or affinity chromatography or, alternatively, it is synthesized by standard recombinant DNA technology in a suitable expression system, such as E. coli, yeast or plants. The tumor-associated antigenic protein is then purified from the expression system by standard chromatographic means. In the case of peptide antigenic components, these are generally prepared by standard automated synthesis. Proteins and peptides can be modified by addition of amino acids, lipids and other agents to improve their incorporation into the delivery system of the vaccine (such as a multilamellar liposome). For a tumor-associated antigenic component derived from the patient's own tumor, or tumors from other individuals, or cell lines, the tumor tissue, or a single cell suspension derived from the tumor tissue, is typically homogenized in a suitable buffer. The homogenate can also be fractionated, such as by centrifugation, to isolate particular cellular components such as cell membranes or soluble material. The tumor material can be used directly or tumor-associated antigens can be extracted for incorporation in the vaccine using a buffer containing a low concentration of a suitable agent such as a detergent. An example of a suitable detergent for extracting antigenic proteins from tumor tissue, tumor cells, and tumor-cell membranes is diheptanoyl phosphatidylcholine. Exosomes derived from tumor tissue or tumor cells, whether autologous or heterologous to the patient, can be used for the antigenic component for incorporation in the vaccine or as a starting material for extraction of tumor-associated antigens.

In some embodiments of the present disclosure, the anti-cancer vaccine is TRICOM: a poxviral-based cancer vaccine (vaccinia Ankara (MVA) replication defective recombinant fowlpox (rF) containing a TRIad of Costimultory Molecules (B7-1, ICAM-1, LFA-3)) (MVA/rF-CEA/TRICOM). Studies have show that cabozantinib in combination of MVA/rF-CEA/TRICOM can alter the phenotype of MC38-CEA murine tumor cells and render the tumor more sensitive to immune-mediated killing. J. Tanslational Medicine, 2014, 12:294.

In some embodiments of the present disclosure, a combination therapy comprises cabozantinib in combination with a cancer vaccine immunotherapeutic agent. In various examples, the cancer vaccine includes at least one tumor-associated antigen, at least one immunostimulant, and optionally, at least one cell-based immunotherapeutic agent. In some embodiments, the immunostimulant component in the cancer vaccine of the disclosure is any Biological Response Modifier (BRM) with the ability to enhance the therapeutic cancer vaccine's effectiveness to induce humoral and cellular immune responses against cancer cells in a patient. According to one aspect, the immunostimulant is a cytokine or combination of cytokines. Examples of such cytokines include the interferons, such as IFN-gamma, the interleukins, such as IL-2, IL-15 and IL-23, the colony stimulating factors, such as M-CSF and GM-CSF, and tumor necrosis factor. According to another aspect, the immunostimulant component of the disclosed cancer vaccine includes one or more adjuvant-type immunostimulatory agents such as APC Toll-like Receptor agonists or costimulatory/cell adhesion membrane proteins, with or without immunostimulatory cytokines. Examples of Toll-like Receptor agonists include lipid A and CpG, and costimulatory/adhesion proteins such as CD80, CD86, and ICAM-1.

In some embodiments, the immunostimulant is selected from the group consisting of IFN-gamma (IFN-γ), IL-2, IL-15, IL-23, M-CSF, GM-CSF, tumor necrosis factor, lipid A, CpG, CD80, CD86, and ICAM-1, or combinations thereof. According to other aspects, the cell-based immunotherapeutic agent is selected from the group consisting of dendritic cells, tumor-infiltrating T lymphocytes, chimeric antigen receptor-modified T effector cells directed to the patient's tumor type, B lymphocytes, natural killer cells, bone marrow cells, and any other cell of a patient's immune system, or combinations thereof. In one aspect, the cancer vaccine immunostimulant includes one or more cytokines, such as interleukin 2 (IL-2), GM-CSF, M-CSF, and interferon-gamma (IFN-γ), one or more Toll-like Receptor agonists and/or adjuvants, such as monophosphoryl lipid A, lipid A, muramyl dipeptide (MDP) lipid conjugate and double stranded RNA, or one or more costimulatory membrane proteins and/or cell adhesion proteins, such CD80, CD86 and ICAM-1, or any combination of the above. In one aspect, the cancer vaccine includes an immunostimulant that is a cytokine selected from the group consisting of interleukin 2 (IL-2), GM-CSF, M-CSF, and interferon-gamma (IFN-γ). In another aspect, the cancer vaccine includes an immunostimulant that is a Toll-like Receptor agonist and/or adjuvant selected from the group consisting of monophosphoryl lipid A, lipid A, and muramyl dipeptide (MDP) lipid conjugate and double stranded RNA. In yet another aspect, the cancer vaccine includes an immunostimulant that is a costimulatory membrane protein and/or cell adhesion protein selected from the group consisting of CD80, CD86, and ICAM-1.

In various embodiments, an immunotherapeutic agent can include a cancer vaccine, wherein the cancer vaccine incorporates any tumor antigen that can be potentially used to construct a fusion protein according to the invention and particularly the following: (a) cancer-testis antigens including NY-ESO-1, SSX2, SCP1 as well as RAGE, BAGE, GAGE and MAGE family polypeptides, for example, GAGE-1, GAGE-2, MAGE-1 MAGE-2, MAGE-3, MAGE-4, MAGE-5, MAGE-6, and MAGE-12, which can be used, for example, to address melanoma, lung, head and neck, NSCLC, breast, gastrointestinal, and bladder tumors; (b) mutated antigens, including p53, associated with various solid tumors, e.g., colorectal, lung, head and neck cancer; p21/Ras associated with, e.g., melanoma, pancreatic cancer and colorectal cancer; CDK4, associated with, e.g., melanoma; MUM1 associated with, e.g., melanoma; caspase-8 associated with, e.g., head and neck cancer; CIA 0205 associated with, e.g., bladder cancer; HLA-A2-R1701, beta catenin associated with, e.g., melanoma; TCR associated with, e.g., T-cell non-Hodgkin lymphoma; BCR-abl associated with, e.g., chronic myelogenous leukemia; triosephosphate isomerase; MA 0205; CDC-27, and LDLR-FUT; (c) over-expressed antigens, including, Galectin 4 associated with, e.g., colorectal cancer; Galectin 9 associated with, e.g., Hodgkin's disease; proteinase 3 associated with, e.g., chronic myelogenous leukemia; WT 1 associated with, e.g., various leukemias; carbonic anhydrase associated with, e.g., renal cancer; aldolase A associated with, e.g., lung cancer; PRAME associated with, e.g., melanoma; HER-2/neu associated with, e.g., breast, colon, lung and ovarian cancer; mammaglobin, alpha-fetoprotein associated with, e.g., hepatoma; KSA associated with, e.g., colorectal cancer; gastrin associated with, e.g., pancreatic and gastric cancer; telomerase catalytic protein, MUC-1 associated with, e.g., breast and ovarian cancer; G-250 associated with, e.g., renal cell carcinoma; p53 associated with, e.g., breast, colon cancer; and carcinoembryonic antigen associated with, e.g., breast cancer, lung cancer, and cancers of the gastrointestinal tract such as colorectal cancer; (d) shared antigens, including melanoma-melanocyte differentiation antigens such as MART-1/Melan A; gp100; MC1R; melanocyte-stimulating hormone receptor; tyrosinase; tyrosinase related protein-1/TRP1 and tyrosinase related protein-2/TRP2 associated with, e.g., melanoma; (e) prostate associated antigens including PAP, PSA, PSMA, PSH-P1, PSM-P1, PSM-P2, associated with e.g., prostate cancer; (f) immunoglobulin idiotypes associated with myeloma and B cell lymphomas. In certain embodiments, the one or more TAA can be selected from pi 5, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, pl 80erbB-3, c-met, mn-23H1, TAG-72-4, CA 19-9, CA 72-4, CAM 17.1, NuMa, K-ras, pi 6, TAGE, PSCA, CT7, 43-9F, 5T4, 791 Tgp72, beta-HCG, BCA225, BTAA, CA 125, CA 15-3 (CA 27.29\BCAA), CA 195, CA 242, CA-50, CAM43, CD68\KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, TPS or any combinations thereof.

In some embodiments, the present disclosure provides cabozantinib for use in combination with a cancer vaccine which can include a tumor antigen comprising the entire amino acid sequence, a portion of it, or specific immunogenic epitopes of a human protein.

In various embodiments, an illustrative immunotherapeutic agent may include an mRNA operable to encode any one or more of the aforementioned cancer antigens useful for synthesizing a cancer vaccine. In some illustrative embodiments, the mRNA based cancer vaccine may have one or more of the following properties: a) the mRNA encoding each cancer antigen is interspersed by cleavage sensitive sites; b) the mRNA encoding each cancer antigen is linked directly to one another without a linker; c) the mRNA encoding each cancer antigen is linked to one another with a single nucleotide linker; d) each cancer antigen comprises a 20-40 amino acids and includes a centrally located SNP mutation; e) at least 40% of the cancer antigens have a highest affinity for class I MHC molecules from the subject; f) at least 40% of the cancer antigens have a highest affinity for class II MHC molecules from the subject; g) at least 40% of the cancer antigens have a predicted binding affinity of IC>500 nM for HLA-A, HLA-B and/or DRB1; h) the mRNA encodes 1 to 15 cancer antigens; i) 10-60% of the cancer antigens have a binding affinity for class I MHC and 10-60% of the cancer antigens have a binding affinity for class II MHC; and/or j) the mRNA encoding the cancer antigens is arranged such that the cancer antigens are ordered to minimize pseudo-epitopes.

In various embodiments, the combination comprising cabozantinib and a cancer vaccine immunotherapeutic agent as disclosed herein can be used to illicit an immune response in a subject against a cancer antigen. The method involves administering to the subject a RNA vaccine comprising at least one RNA polynucleotide having an open reading frame encoding at least one antigenic polypeptide or an immunogenic fragment thereof, thereby inducing in the subject an immune response specific to the antigenic polypeptide or an immunogenic fragment thereof, in combination with administering cabozantinib either in the same composition or a separate composition, administered at the same time, or sequentially dosed, wherein the anti-antigenic polypeptide antibody titer in the subject is increased following vaccination relative to anti-antigenic polypeptide antibody titer in a subject vaccinated with a prophylactically effective dose of a traditional vaccine against the cancer. An “anti-antigenic polypeptide antibody” is a serum antibody the binds specifically to the antigenic polypeptide.

A prophylactically effective dose is a therapeutically effective dose that prevents advancement of cancer at a clinically acceptable level. In some embodiments the therapeutically effective dose is a dose listed in a package insert for the vaccine. A traditional vaccine, as used herein, refers to a vaccine other than the mRNA vaccines of the invention. For instance, a traditional vaccine includes but is not limited to live microorganism vaccines, killed microorganism vaccines, subunit vaccines, protein antigen vaccines, DNA vaccines, and the like. In exemplary embodiments, a traditional vaccine is a vaccine that has achieved regulatory approval and/or is registered by a national drug regulatory body, for example the Food and Drug Administration (FDA) in the United States or the European Medicines Agency (EMA.)

In some embodiments the anti-antigenic polypeptide antibody titer in the subject is increased 1 log to 10 log following vaccination relative to anti-antigenic polypeptide antibody titer in a subject vaccinated with a prophylactically effective dose of a traditional vaccine against the cancer. In some embodiments the anti-antigenic polypeptide antibody titer in the subject is increased 1 log following vaccination relative to anti-antigenic polypeptide antibody titer in a subject vaccinated with a prophylactically effective dose of a traditional vaccine against the cancer. In some embodiments the anti-antigenic polypeptide antibody titer in the subject is increased 2 log following vaccination relative to anti-antigenic polypeptide antibody titer in a subject vaccinated with a prophylactically effective dose of a traditional vaccine against the cancer.

Aspects of the invention provide nucleic acid vaccines comprising one or more RNA polynucleotides having an open reading frame encoding a first antigenic polypeptide, wherein the RNA polynucleotide is present in the formulation for in vivo administration to a host, which confers an antibody titer superior to the criterion for sero-protection for the first antigen for an acceptable percentage of human subjects. In some embodiments, the antibody titer produced by the mRNA vaccines of the invention is a neutralizing antibody titer. In some embodiments the neutralizing antibody titer is greater than a protein vaccine. In other embodiments the neutralizing antibody titer produced by the mRNA vaccines of the invention is greater than an adjuvanted protein vaccine. In yet other embodiments the neutralizing antibody titer produced by the mRNA vaccines of the invention is 1,000-10,000, 1,200-10,000, 1,400-10,000, 1,500-10,000, 1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typically expressed as the highest serum dilution required to achieve a 50% reduction in the number of plaques.

In preferred aspects, RNA vaccine immunotherapeutic agents of the present disclosure (e.g., mRNA vaccines) produce prophylactically- and/or therapeutically-efficacious levels, concentrations and/or titers of antigen-specific antibodies in the blood or serum of a vaccinated subject. As defined herein, the term antibody titer refers to the amount of antigen-specific antibody produced in a subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In certain aspects, antibody titer measurement is expressed as a ratio, such as 1:40, 1:100, and the like.

In exemplary embodiments of the invention, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.) In exemplary aspects of the invention, antigen-specific antibodies are measured in units of g/ml or are measured in units of IU/L (International Units per liter) or mIU/ml (milli International Units per ml). In exemplary embodiments of the invention, an efficacious vaccine produces >0.5 ng/mL, >0.1 ng/mL, >0.2 ng/mL, >0.35 ng/mL, >0.5 ng/mL, >1 ng/mL, >2 ng/mL, >5 ng/mL or >10 ng/mL. In exemplary embodiments of the invention, an efficacious vaccine produces >10 mIU/mL, >20 mIU/mL, >50 mIU/mL, >100 mIU/mL, >200 mIU/mL, >500 mIU/ml or >1000 mIU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay. Also provided are nucleic acid vaccines comprising one or more RNA polynucleotides having an open reading frame encoding a first antigenic polypeptide or a concatemeric polypeptide, wherein the RNA polynucleotide is present in a formulation for in vivo administration to a host for eliciting a longer lasting high antibody titer than an antibody titer elicited by an mRNA vaccine having a stabilizing element or formulated with an adjuvant and encoding the first antigenic polypeptide. In some embodiments, the RNA polynucleotide is formulated to produce neutralizing antibodies within one week of a single administration. In some embodiments, the adjuvant is selected from a cationic peptide and an immunostimulatory nucleic acid. In some embodiments, the cationic peptide is protamine.

Immunotherapeutic agents comprising a nucleic acid vaccine comprising one or more RNA polynucleotides having an open reading frame comprising at least one chemical modification or optionally no nucleotide modification, the open reading frame encoding a first antigenic polypeptide or a concatemeric polypeptide, wherein the RNA polynucleotide is present in the formulation for in vivo administration to a host such that the level of antigen expression in the host significantly exceeds a level of antigen expression produced by an mRNA vaccine having a stabilizing element or formulated with an adjuvant and encoding the first antigenic polypeptide.

Other aspects provide nucleic acid vaccines comprising one or more RNA polynucleotides having an open reading frame comprising at least one chemical modification or optionally no nucleotide modification, the open reading frame encoding a first antigenic polypeptide or a concatemeric polypeptide, wherein the vaccine has at least 10 fold less RNA polynucleotide than is required for an unmodified mRNA vaccine to produce an equivalent antibody titer. In some embodiments, the RNA polynucleotide is present in a dosage of 25-100 micrograms.

Aspects of the invention also provide a unit of use vaccine, comprising between 10 ng and 400 ng of one or more RNA polynucleotides having an open reading frame comprising at least one chemical modification or optionally no nucleotide modification, the open reading frame encoding a first antigenic polypeptide or a concatemeric polypeptide, and a pharmaceutically acceptable excipient, formulated for delivery to a human subject. In some embodiments, the vaccine further comprises a cationic lipid nanoparticle.

Aspects of the invention provide methods of creating, maintaining or restoring antigenic memory to a tumor in an individual or population of individuals comprising administering to said individual or population an antigenic memory booster nucleic acid vaccine comprising (a) at least one RNA polynucleotide, said polynucleotide comprising at least one chemical modification or optionally no nucleotide modification and two or more codon-optimized open reading frames, said open reading frames encoding a set of reference antigenic polypeptides, and (b) optionally a pharmaceutically acceptable excipient. In some embodiments, the vaccine is administered to the individual via a route selected from the group consisting of intramuscular administration, intradermal administration and subcutaneous administration. In some embodiments, the administering step comprises contacting a muscle tissue of the subject with a device suitable for injection of the composition. In some embodiments, the administering step comprises contacting a muscle tissue of the subject with a device suitable for injection of the composition in combination with electroporation.

Aspects of the invention provide methods of vaccinating a subject comprising administering to the subject a single dosage of between 25 ng/kg and 400 ng/kg of a nucleic acid vaccine comprising one or more RNA polynucleotides having an open reading frame encoding a first antigenic polypeptide or a concatemeric polypeptide in an effective amount to vaccinate the subject.

Other aspects provide nucleic acid vaccines comprising one or more RNA polynucleotides having an open reading frame comprising at least one chemical modification, the open reading frame encoding a first antigenic polypeptide or a concatemeric polypeptide, wherein the vaccine has at least 10 fold less RNA polynucleotide than is required for an unmodified mRNA vaccine to produce an equivalent antibody titer. In some embodiments, the RNA polynucleotide is present in a dosage of 25-100 micrograms.

### EXAMPLES

### Preparation of Cabozantinib S-Malate

### Preparation of N-(4-{[6,7-bis(methyloxy)quinolin-4-yl]oxy}phenyl)-N′-(4-fluorophenyl)cyclopropane-1,1-dicarboxamide and the (L)-malate salt thereof

The synthetic route used for the preparation of N-(4-{[6,7-bis(methyloxy)quinolin-4-yl]oxy}phenyl)-N′-(4-fluorophenyl)cyclopropane-1,1-dicarboxamide and the (L)-malate salt thereof is depicted in Scheme 1:

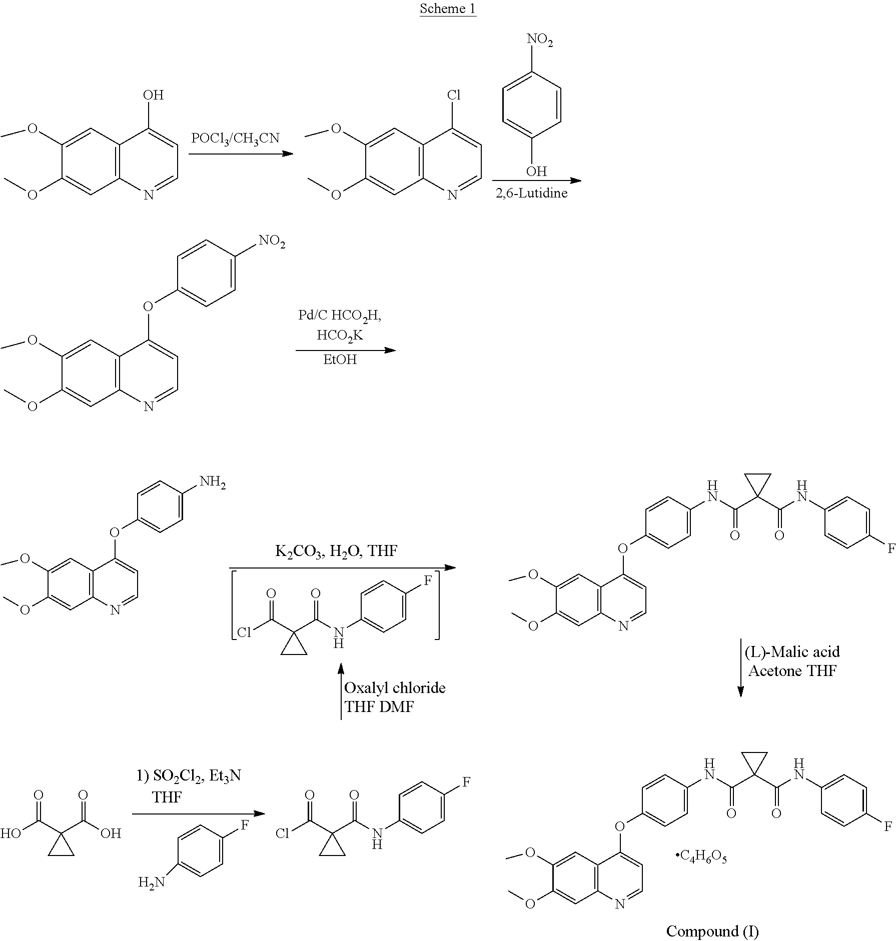

### Preparation of 4-Chloro-6,7-dimethoxy-quinoline

A reactor was charged sequentially with 6,7-dimethoxy-quinoline-4-ol (10.0 kg) and acetonitrile (64.0 L). The resulting mixture was heated to approximately 65° C. and phosphorus oxychloride (POCl₃, 50.0 kg) was added. After the addition of POCl₃, the temperature of the reaction mixture was raised to approximately 80° C. The reaction was deemed complete (approximately 9.0 hours) when less than 2 percent of the starting material remained (in process high-performance liquid chromotography [HPLC] analysis). The reaction mixture was cooled to approximately 10° C. and then quenched into a chilled solution of dichloromethane (DCM, 238.0 kg), 30% NH₄OH (135.0 kg), and ice (440.0 kg). The resulting mixture was warmed to approximately 14° C., and phases were separated. The organic phase was washed with water (40.0 kg) and concentrated by vacuum distillation to remove the solvent (approximately 190.0 kg). Methyl-t-butyl ether (MTBE, 50.0 kg) was added to the batch, and the mixture was cooled to approximately 10° C., during which time the product crystallized out. The solids were recovered by centrifugation, washed with n heptane (20.0 kg), and dried at approximately 40° C. to afford the title compound (8.0 kg).

### Preparation of 6,7-Dimethyl-4-(4-nitro-phenoxy)-quinoline

A reactor was sequentially charged with 4-chloro-6,7-dimethoxy-quinoline (8.0 kg), 4 nitrophenol (7.0 kg), 4 dimethylaminopyridine (0.9 kg), and 2,6 lutidine (40.0 kg). The reactor contents were heated to approximately 147° C. When the reaction was complete (less than 5 percent starting material remaining as determined by in process HPLC analysis, approximately 20 hours), the reactor contents were allowed to cool to approximately 25° C. Methanol (26.0 kg) was added, followed by potassium carbonate (3.0 kg) dissolved in water (50.0 kg). The reactor contents were stirred for approximately 2 hours. The resulting solid precipitate was filtered, washed with water (67.0 kg), and dried at 25° C. for approximately 12 hours to afford the title compound (4.0 kg).

### Preparation of 4-(6,7-Dimethoxy-quinoline-4-yloxy)-phenylamine

A solution containing potassium formate (5.0 kg), formic acid (3.0 kg), and water (16.0 kg) was added to a mixture of 6,7-dimethoxy-4-(4-nitro-phenoxy)-quinoline (4.0 kg), 10 percent palladium on carbon (50 percent water wet, 0.4 kg) in tetrahydrofuran (THF, 40.0 kg) that had been heated to approximately 60° C. The addition was carried out such that the temperature of the reaction mixture remained approximately 60° C. When the reaction was deemed complete as determined using in-process HPLC analysis (less than 2 percent starting material remaining, typically 1 5 hours), the reactor contents were filtered. The filtrate was concentrated by vacuum distillation at approximately 35° C. to half of its original volume, which resulted in the precipitation of the product. The product was recovered by filtration, washed with water (12.0 kg), and dried under vacuum at approximately 50° C. to afford the title compound (3.0 kg; 97 percent area under curve (AUC)).

### Preparation of 1-(4-Fluoro-phenylcarbamoyl)-cyclopropanecarboxylic acid

Triethylamine (8.0 kg) was added to a cooled (approximately 4° C.) solution of commercially available cyclopropane-1,1-dicarboxylic acid (2 1, 10.0 kg) in THF (63.0 kg) at a rate such that the batch temperature did not exceed 10° C. The solution was stirred for approximately 30 minutes, and then thionyl chloride (9.0 kg) was added, keeping the batch temperature below 10° C. When the addition was complete, a solution of 4-fluoroaniline (9.0 kg) in THF (25.0 kg) was added at a rate such that the batch temperature did not exceed 10° C. The mixture was stirred for approximately 4 hours and then diluted with isopropyl acetate (87.0 kg). This solution was washed sequentially with aqueous sodium hydroxide (2.0 kg dissolved in 50.0 L of water), water (40.0 L), and aqueous sodium chloride (10.0 kg dissolved in 40.0 L of water). The organic solution was concentrated by vacuum distillation followed by the addition of heptane, which resulted in the precipitation of solid. The solid was recovered by centrifugation and then dried at approximately 35° C. under vacuum to afford the title compound. (10.0 kg).

### Preparation of 1-(4-Fluoro-phenylcarbamoyl)-cyclopropanecarbonyl chloride

Oxalyl chloride (1.0 kg) was added to a solution of 1-(4-fluoro-phenylcarbamoyl)-cyclopropanecarboxylic acid (2.0 kg) in a mixture of THF (11 kg) and N, N-dimethylformamide (DMF; 0.02 kg) at a rate such that the batch temperature did not exceed 30° C. This solution was used in the next step without further processing.

### Preparation of N-(4-{[6,7-bis(methyloxy)quinolin-4-yl]oxy}phenyl)-N′-(4-fluorophenyl)cyclopropane-1,1-dicarboxamide

The solution from the previous step containing 1-(4-fluoro-phenylcarbamoyl)-cyclopropanecarbonyl chloride was added to a mixture of 4-(6,7-dimethoxy-quinoline-4-yloxy)-phenylamine (3.0 kg) and potassium carbonate (4.0 kg) in THF (27.0 kg) and water (13.0 kg) at a rate such that the batch temperature did not exceed 30° C. When the reaction was complete (in typically 10 minutes), water (74.0 kg) was added. The mixture was stirred at 15-30° C. for approximately 10 hours, which resulted in the precipitation of the product. The product was recovered by filtration, washed with a pre-made solution of THF (11.0 kg) and water (24.0 kg), and dried at approximately 65° C. under vacuum for approximately 12 hours to afford the title compound (free base, 5.0 kg). ¹H NMR (400 MHz, d₆-DMSO): δ 10.2 (s, 1H), 10.05 (s, 1H), 8.4 (s, 1H), 7.8 (m, 2H), 7.65 (m, 2H), 7.5 (s, 1H), 7.35 (s, 1H), 7.25 (m, 2H), 7.15 (m, 2H), 6.4 (s, 1H), 4.0 (d, 6H), 1.5 (s, 4H). LC/MS: M+H=502.

### Preparation of N-(4-{[6,7-bis(methyloxy)quinolin-4-yl]oxy}phenyl)-N′-(4-fluorophenyl)cyclopropane-1,1-dicarboxamide, (L) malate salt

A solution of L-malic acid (2.0 kg) in water (2.0 kg) was added to a solution of Cyclopropane-1,1-dicarboxylic acid [4-(6,7-dimethoxy-quinoline-4-yloxy)-phenyl]-amide (4-fluoro-phenyl)-amide free base (1 5, 5.0 kg) in ethanol, maintaining a batch temperature of approximately 25° C. Carbon (0.5 kg) and thiol silica (0.1 kg) were then added, and the resulting mixture was heated to approximately 78° C., at which point water (6.0 kg) was added. The reaction mixture was then filtered, followed by the addition of isopropanol (38.0 kg), and was allowed to cool to approximately 25° C. The product was recovered by filtration and washed with isopropanol (20.0 kg), and dried at approximately 65° C. to afford the title compound (5.0 kg).

### Alternative Preparation of N-(4-{[6,7-Bis(methyloxy)quinolin-4-yl]oxy}phenyl)-N′-(4-fluorophenyl)cyclopropane-1,1-dicarboxamide and the (L)-malate salt thereof

An alternative synthetic route that can be used for the preparation of N-(4-{[6,7-bis(methyloxy)quinolin-4-yl]oxy}phenyl)-N′-(4-fluorophenyl)cyclopropane-1,1-dicarboxamide and the (L)-malate salt thereof is depicted in Scheme 2, as described in PCT/US2012/024591, the entire contents of which is incorporated by reference.

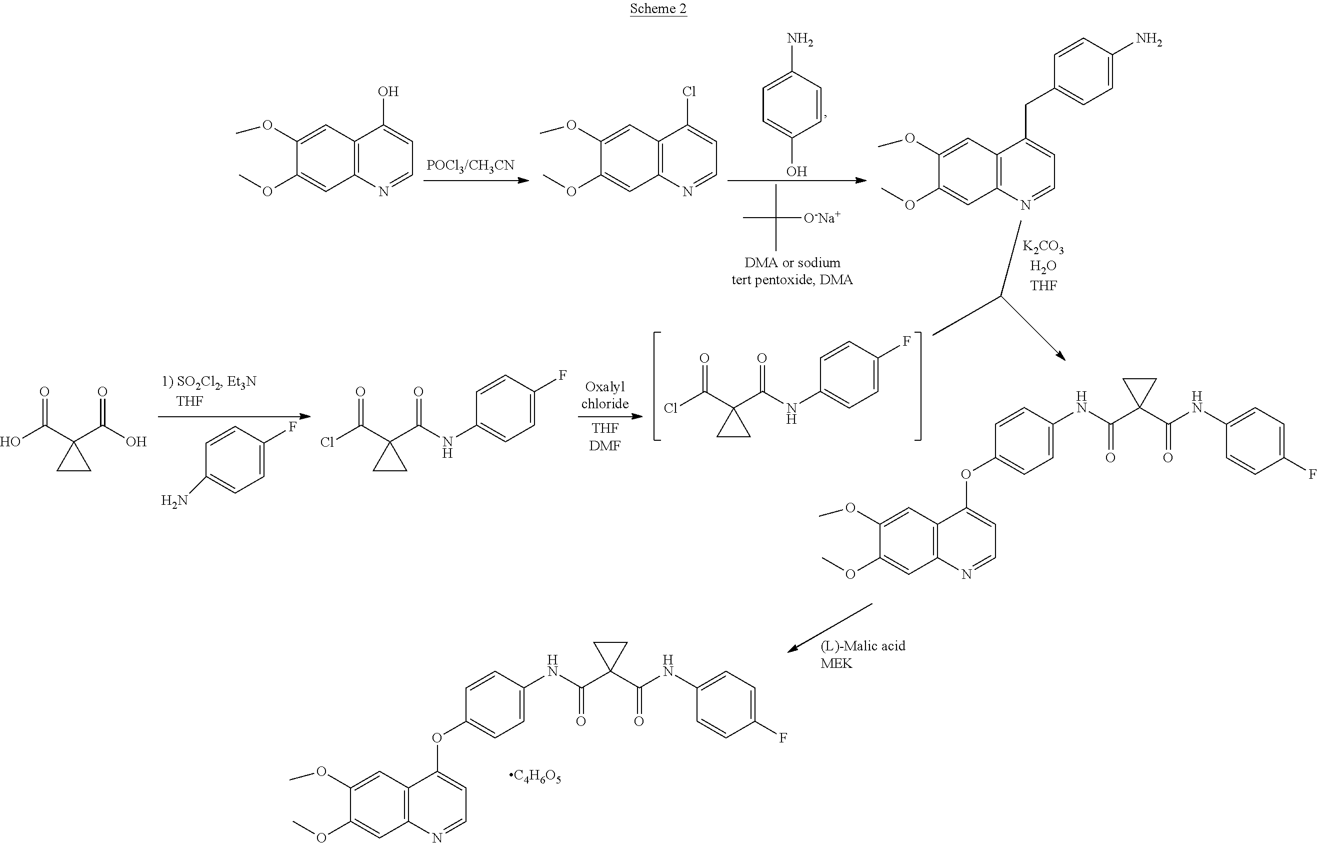

### Preparation of 4-Chloro-6,7-dimethoxy-quinoline

A reactor was charged sequentially with 6,7-dimethoxy-quinoline-4-ol (47.0 kg) and acetonitrile (318.8 kg). The resulting mixture was heated to approximately 60° C. and phosphorus oxychloride (POCl₃, 130.6 kg) was added. After the addition of POCl₃, the temperature of the reaction mixture was raised to approximately 77° C. The reaction was deemed complete (approximately 13 hours) when less than 3% of the starting material remained (in-process high-performance liquid chromatography [HPLC] analysis). The reaction mixture was cooled to approximately 2-7° C. and then quenched into a chilled solution of dichloromethane (DCM, 482.8 kg), 26 percent NH₄OH (251.3 kg), and water (900 L). The resulting mixture was warmed to approximately 20-25° C., and phases were separated. The organic phase was filtered through a bed of AW hyflo super-cel NF (Celite; 5.4 kg) and the filter bed was washed with DCM (118.9 kg). The combined organic phase was washed with brine (282.9 kg) and mixed with water (120 L). The phases were separated and the organic phase was concentrated by vacuum distillation with the removal of solvent (approximately 95 L residual volume). DCM (686.5 kg) was charged to the reactor containing organic phase and concentrated by vacuum distillation with the removal of solvent (approximately 90 L residual volume). Methyl t-butyl ether (MTBE, 226.0 kg) was then charged and the temperature of the mixture was adjusted to −20 to −25° C. and held for 2.5 hours resulting in solid precipitate which was then filtered and washed with n-heptane (92.0 kg), and dried on a filter at approximately 25° C. under nitrogen to afford the title compound. (35.6 kg).

### Preparation of 4-(6,7-Dimethoxy-quinoline-4-yloxy)-phenylamine

4-Aminophenol (24.4 kg) dissolved in N,N-dimethylacetamide (DMA, 184.3 kg) was charged to a reactor containing 4-chloro-6,7-dimethoxyquinoline (35.3 kg), sodium t-butoxide (21.4 kg) and DMA (167.2 kg) at 20-25° C. This mixture was then heated to 100-105° C. for approximately 13 hours. After the reaction was deemed complete as determined using in-process HPLC analysis (less than 2 percent starting material remaining), the reactor contents were cooled at 15-20° C. and water (pre-cooled, 2-7° C., 587 L) charged at a rate to maintain 15-30° C. temperature. The resulting solid precipitate was filtered, washed with a mixture of water (47 L) and DMA (89.1 kg) and finally with water (214 L). The filter cake was then dried at approximately 25° C. on filter to yield crude 4-(6, 7-dimethoxy-quinoline-4-yloxy)-phenylamine (59.4 kg wet, 41.6 kg dry calculated based on LOD). Crude 4-(6, 7-dimethoxy-quinoline-4-yloxy)-phenylamine was refluxed (approximately 75° C.) in a mixture of tetrahydrofuran (THF, 211.4 kg) and DMA (108.8 kg) for approximately 1 hour and then cooled to 0-5° C. and aged for approximately 1 hour after which time the solid was filtered, washed with THF (147.6 kg) and dried on a filter under vacuum at approximately 25° C. to yield 4-(6,7-dimethoxy-quinoline-4-yloxy)-phenylamine (34.0 kg).

### Alternative Preparation of 4-(6, 7-Dimethoxy-quinoline-4-yloxy)-phenylamine

4-chloro-6,7-dimethoxyquinoline (34.8 kg) and 4-aminophenol (30.8 kg) and sodium tert pentoxide (1.8 equivalents) 88.7 kg, 35 weight percent in THF) were charged to a reactor, followed by N,N-dimethylacetamide (DMA, 293.3 kg). This mixture was then heated to 105-115° C. for approximately 9 hours. After the reaction was deemed complete as determined using in-process HPLC analysis (less than 2 percent starting material remaining), the reactor contents were cooled at 15-25° C. and water (315 kg) was added over a two hour period while maintaining the temperature between 20-30° C. The reaction mixture was then agitated for an additional hour at 20-25° C. The crude product was collected by filtration and washed with a mixture of 88 kg water and 82.1 kg DMA, followed by 175 kg water. The product was dried on a filter drier for 53 hours. The LOD showed less than 1 percent w/w.

In an alternative procedure, 1.6 equivalents of sodium tert-pentoxide were used and the reaction temperature was increased from 110-120° C. In addition, the cool down temperature was increased to 35-40° C. and the starting temperature of the water addition was adjusted to 35-40° C., with an allowed exotherm to 45° C.

### Preparation of 1-(4-Fluoro-phenylcarbamoyl)-cyclopropanecarboxylic acid

Triethylamine (19.5 kg) was added to a cooled (approximately 5° C.) solution of cyclopropane-1,1-dicarboxylic acid (24.7 kg) in THF (89.6 kg) at a rate such that the batch temperature did not exceed 5° C. The solution was stirred for approximately 1.3 hours, and then thionyl chloride (23.1 kg) was added, keeping the batch temperature below 10° C. When the addition was complete, the solution was stirred for approximately 4 hours keeping temperature below 10° C. A solution of 4-fluoroaniline (18.0 kg) in THF (33.1 kg) was then added at a rate such that the batch temperature did not exceed 10° C. The mixture was stirred for approximately 10 hours after which the reaction was deemed complete. The reaction mixture was then diluted with isopropyl acetate (218.1 kg). This solution was washed sequentially with aqueous sodium hydroxide (10.4 kg, 50 percent dissolved in 119 L of water) further diluted with water (415 L), then with water (100 L) and finally with aqueous sodium chloride (20.0 kg dissolved in 100 L of water). The organic solution was concentrated by vacuum distillation (100 L residual volume) below 40° C. followed by the addition of n-heptane (171.4 kg), which resulted in the precipitation of solid. The solid was recovered by filtration and washed with n-heptane (102.4 kg), resulting in wet, crude 1-(4-fluoro-phenylcarbamoyl)-cyclopropanecarboxylic acid (29.0 kg). The crude, 1-(4-fluoro-phenylcarbamoyl)-cyclopropanecarboxylic acid was dissolved in methanol (139.7 kg) at approximately 25° C. followed by the addition of water (320 L) resulting in slurry which was recovered by filtration, washed sequentially with water (20 L) and n-heptane (103.1 kg) and then dried on the filter at approximately 25° C. under nitrogen to afford the title compound (25.4 kg).

### Preparation of 1-(4-Fluoro-phenylcarbamoyl)-cyclopropanecarbonyl chloride

Oxalyl chloride (12.6 kg) was added to a solution of 1-(4-fluoro-phenylcarbamoyl)-cyclopropanecarboxylic acid (22.8 kg) in a mixture of THF (96.1 kg) and N, N-dimethylformamide (DMF; 0.23 kg) at a rate such that the batch temperature did not exceed 25° C. This solution was used in the next step without further processing.

### Alternative Preparation of 1-(4-Fluoro-phenylcarbamoyl)-cyclopropanecarbonyl chloride

A reactor was charged with 1-(4-fluoro-phenylcarbamoyl)-cyclopropanecarboxylic acid (35 kg), 344 g DMF, and 175 kg THF. The reaction mixture was adjusted to 12-17° C. and then to the reaction mixture was charged 19.9 kg of oxalyl chloride over a period of 1 hour. The reaction mixture was left stirring at 12-17° C. for 3 to 8 hours. This solution was used in the next step without further processing.

### Preparation of cyclopropane-1,1-dicarboxylic acid [4-(6,7-dimethoxy-quinoline-4-yloxy)-phenyl]-amide (4-fluoro-phenyl)-amide

The solution from the previous step containing 1-(4-fluoro-phenylcarbamoyl)-cyclopropanecarbonyl chloride was added to a mixture of compound 4-(6,7-dimethoxy-quinoline-4-yloxy)-phenylamine (23.5 kg) and potassium carbonate (31.9 kg) in THF (245.7 kg) and water (116 L) at a rate such that the batch temperature did not exceed 30° C. When the reaction was complete (in approximately 20 minutes), water (653 L) was added. The mixture was stirred at 20-25° C. for approximately 10 hours, which resulted in the precipitation of the product. The product was recovered by filtration, washed with a pre-made solution of THF (68.6 kg) and water (256 L), and dried first on a filter under nitrogen at approximately 25° C. and then at approximately 45° C. under vacuum to afford the title compound (41.0 kg, 38.1 kg, calculated based on LOD).

### Alternative Preparation of cyclopropane-1,1-dicarboxylic acid [4-(6,7-dimethoxy-quinoline-4-yloxy)-phenyl]-amide (4-fluoro-phenyl)-amide

A reactor was charged with 4-(6,7-dimethoxy-quinoline-4-yloxy)-phenylamine (35.7 kg, 1 equivalent), followed by 412.9 kg THF. To the reaction mixture was charged a solution of 48.3 K₂CO₃ in 169 kg water. The acid chloride solution of described in the Alternative Preparation of 1-(4-Fluoro-phenylcarbamoyl)-cyclopropanecarbonyl chloride above was transferred to the reactor containing 4-(6,7-dimethoxy-quinoline-4-yloxy)-phenylamine while maintaining the temperature between 20-30° C. over a minimum of two hours. The reaction mixture was stirred at 20-25° C. for a minimum of three hours. The reaction temperature was then adjusted to 30-25° C. and the mixture was agitated. The agitation was stopped and the phases of the mixture were allowed to separate. The lower aqueous phase was removed and discarded. To the remaining upper organic phase was added 804 kg water. The reaction was left stirring at 15-25° C. for a minimum of 16 hours.

The product precipitated. The product was filtered and washed with a mixture of 179 kg water and 157.9 kg THF in two portions. The crude product was dried under a vacuum for at least two hours. The dried product was then taken up in 285.1 kg THF. The resulting suspension was transferred to reaction vessel and agitated until the suspension became a clear (dissolved) solution, which required heating to 30-35° C. for approximately 30 minutes. 456 kg water was then added to the solution, as well as 20 kg SDAG-1 ethanol (ethanol denatured with methanol over two hours. The mixture was agitated at 15-25° C. fir at least 16 hours. The product was filtered and washed with a mixture of 143 kg water and 126.7 THF in two portions. The product was dried at a maximum temperature set point of 40° C.

In an alternative procedure, the reaction temperature during acid chloride formation was adjusted to 10-15° C. The recrystallization temperature was changed from 15-25° C. to 45-50° C. for 1 hour and then cooled to 15-25° C. over 2 hours.

### Preparation of cyclopropane-1,1-dicarboxylic acid [4-(6,7-dimethoxy-quinoline-4-yloxy)-phenyl]-amide (4-fluoro-phenyl)-amide, malate salt

Cyclopropane-1,1-dicarboxylic acid [4-(6,7-dimethoxy-quinoline-4-yloxy)-phenyl]— amide (4-fluoro-phenyl)-amide (1-5; 13.3 kg), L-malic acid (4.96 kg), methyl ethyl ketone (MEK; 188.6 kg) and water (37.3 kg) were charged to a reactor and the mixture was heated to reflux (approximately 74° C.) for approximately 2 hours. The reactor temperature was reduced to 50 to 55° C. and the reactor contents were filtered. These sequential steps described above were repeated two more times starting with similar amounts of starting material (13.3 kg), L-Malic acid (4.96 kg), MEK (198.6 kg) and water (37.2 kg). The combined filtrate was azeotropically dried at atmospheric pressure using MEK (1133.2 kg) (approximate residual volume 711 L; KF ≤0.5% w/w) at approximately 74° C. The temperature of the reactor contents was reduced to 20 to 25° C. and held for approximately 4 hours resulting in solid precipitate which was filtered, washed with MEK (448 kg) and dried under vacuum at 50° C. to afford the title compound (45.5 kg).

### Alternative Preparation of cyclopropane-1,1-dicarboxylic acid [4-(6,7-dimethoxy-quinoline-4-yloxy)-phenyl]-amide (4-fluoro-phenyl)-amide, (L) malate salt

Cyclopropane-1,1-dicarboxylic acid [4-(6,7-dimethoxy-quinoline-4-yloxy)-phenyl]-amide (4-fluoro-phenyl)-amide (47.9 kg), L-malic acid (17.2), 658.2 kg methyl ethyl ketone, and 129.1 kg water (37.3 kg) were charged to a reactor and the mixture was heated 50-55° C. for approximately 1-3 hours, and then at 55-60° C. for an addition al 4-5 hours. The mixture was clarified by filtration through a 1 μm cartridge. The reactor temperature was adjusted to 20-25° C. and vacuum distilled with a vacuum at 150-200 mm Hg with a maximum jacket temperature of 55° C. to the volume range of 558-731 L.

The vacuum distillation was performed two more times with the charge of 380 kg and 380.2 kg methyl ethyl ketone, respectively. After the third distillation, the volume of the batch was adjusted to 18 v/w of cyclopropane-1,1-dicarboxylic acid [4-(6,7-dimethoxy-quinoline-4-yloxy)-phenyl]-amide (4-fluoro-phenyl)-amide by charging 159.9 kg methyl ethyl ketone to give a total volume of 880L. An addition al vacuum distillation was carried out by adjusting 245.7 methyl ethyl ketone. The reaction mixture was left with moderate agitation at 20-25° C. for at least 24 hours. The product was filtered and washed with 415.1 kg methyl ethyl ketone in three portions. The product was dried under a vacuum with the jacket temperature set point at 45° C.

In an alternative procedure, the order of addition was changed so that a solution of 17.7 kg L-malic acid dissolved in 129.9 kg water was added to cyclopropane-1,1-dicarboxylic acid [4-(6,7-dimethoxy-quinoline-4-yloxy)-phenyl]-amide (4-fluoro-phenyl)-amide (48.7 kg) in methyl ethyl ketone (673.3 kg).

### Biological Example

Tumor tissue is harvested from a subject having a clear cell renal cell carcinoma. The tissue is analyzed according to standard IHC methods to measure position and presence of specific cell types using antibodies that target cell specific markers. The expression levels of cell types are analyzed including intensity and area. Validated assay staining parameters are used for both CD68 and CD8 cells. FIG. 1 shows that the phenotypes of the patient's tumor sample can be used to predict the response of the patient to an anti-tumor therapy. The “T Cell Rich” phenotype is associated with greater tumor shrinkage from baseline and overall response after combination therapy using cabozantinib with the immune checkpoint inhibitor atezolizumab.

### IHC Analyses

Immunohistochemistry analyses to evaluate the relative levels of MET, AXL, VEGFR2, CD31, CD8, CD68, and PD-L1 in archival or fresh FFPE tumor tissue were performed at Covance Inc (Morrisville, NC and Los Angeles, CA). Staining was done using standard methodology for heat induced epitope retrieval (HEIR) followed by primary antibody incubation, washing and then ultraVIEW DAB detection (Ventana). Antibodies clones used for evaluation of tumor tissue include SP44 (MET), C89E7 (AXL), 55B11 (VEGFR2), JC70 (CD31), CD8, CD68, and SP142 (PD-L1).

Categorical quantification of CD8 and CD68 density (frequency or level) was assigned as the percentage of positive staining cells: High 20%, Low <20%. PD-L1 scoring was positive for immune cells/area of 1% and negative as ≤1%. Combination of CD8 with PD-L1 was determined as: Hot—PD-L1 positive and/or CD8 High, Cold—CD8 low and PD-L1 negative. The composite categorical score for CD8 with CD68 was: Hot—CD8 high and CD68 high, Myeloid dominant—CD68 high and CD8 low, and Immune cold—CD68 low and CD8 low.

H-scores were generated (%1+x 1)+(%2+x 2)+(%3+x 3) for MET, AXL and VEGFR2 tumor cell membrane staining. Additionally, categorical calls for MET and AXL membrane staining were established as High 50% tumor cells positive, Low 49%-1%, and Negative as no staining. Subsequent imaging analyses enumerating CD8 and CD68 cell density per all nucleated cells was performed at Flagship Biosciences (Westminster, CO). For digital analysis, tumor and stromal areas were manually annotated by a pathologist. Cell nuclei were identified from the hematoxylin counterstain and positive cells from DAB using flagship algorithms.

Association of PD-L1, CD8, and CD68 with Response and Lesion Change in ccRCC and nccRCC Baseline Tumor Tissue

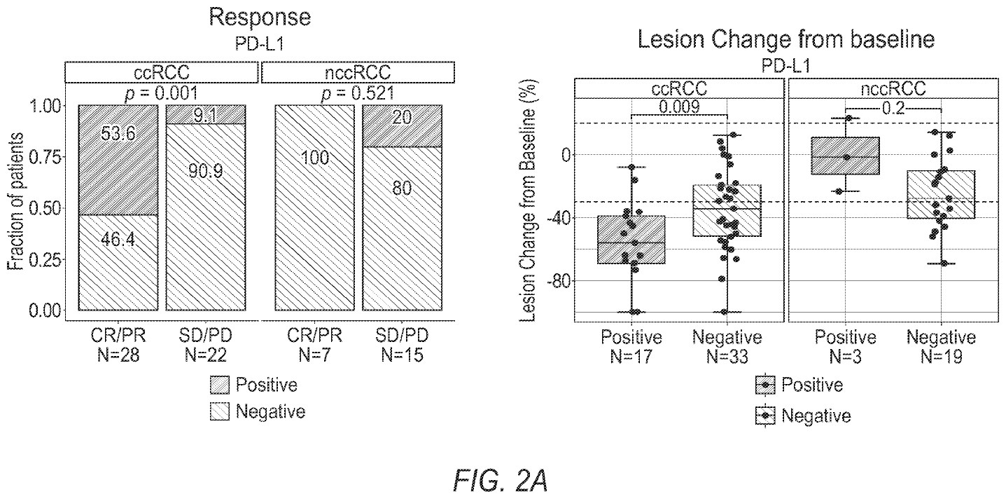

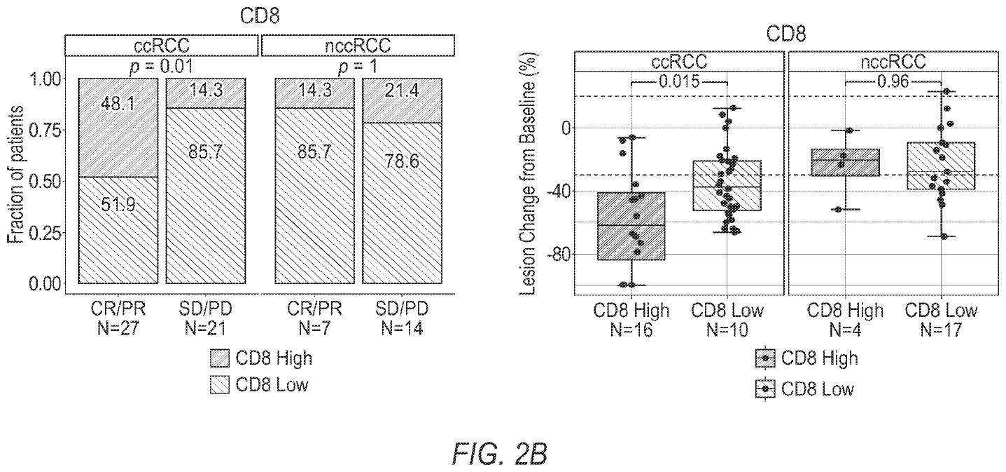

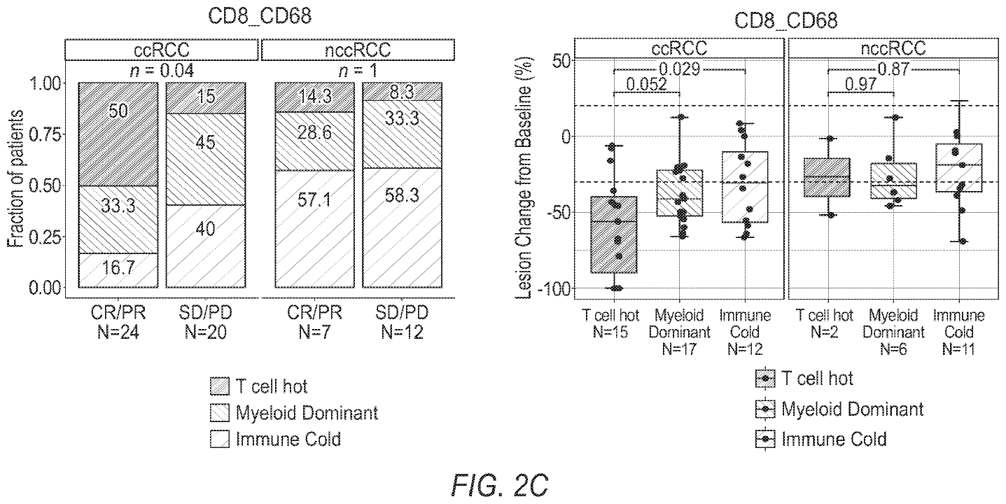

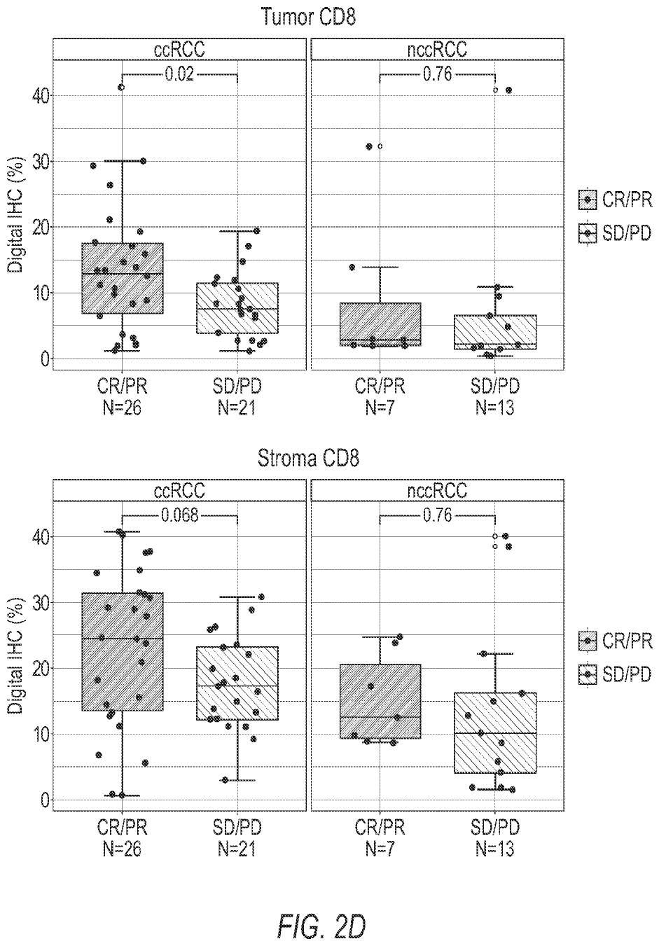

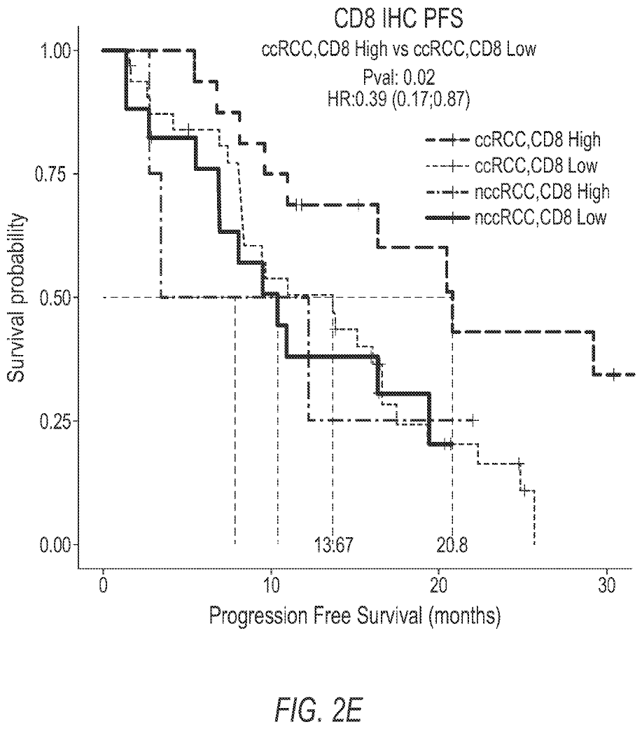

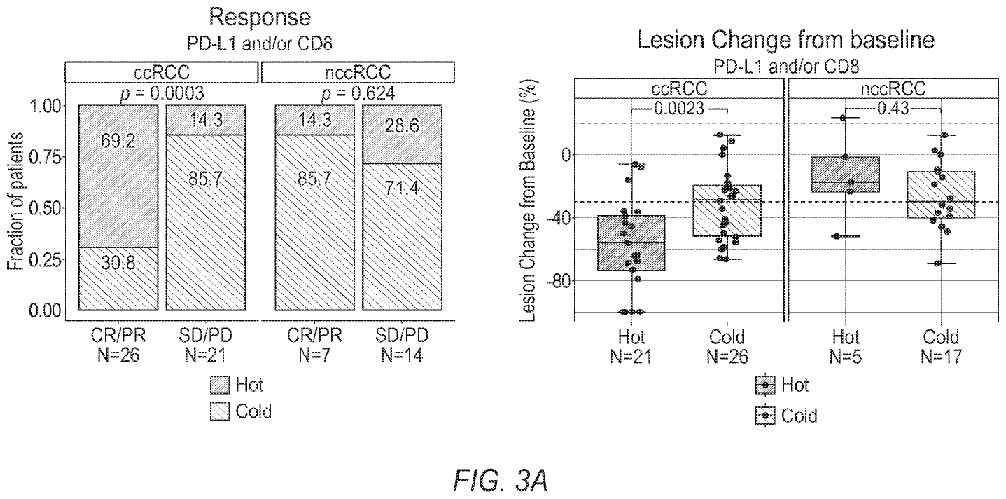

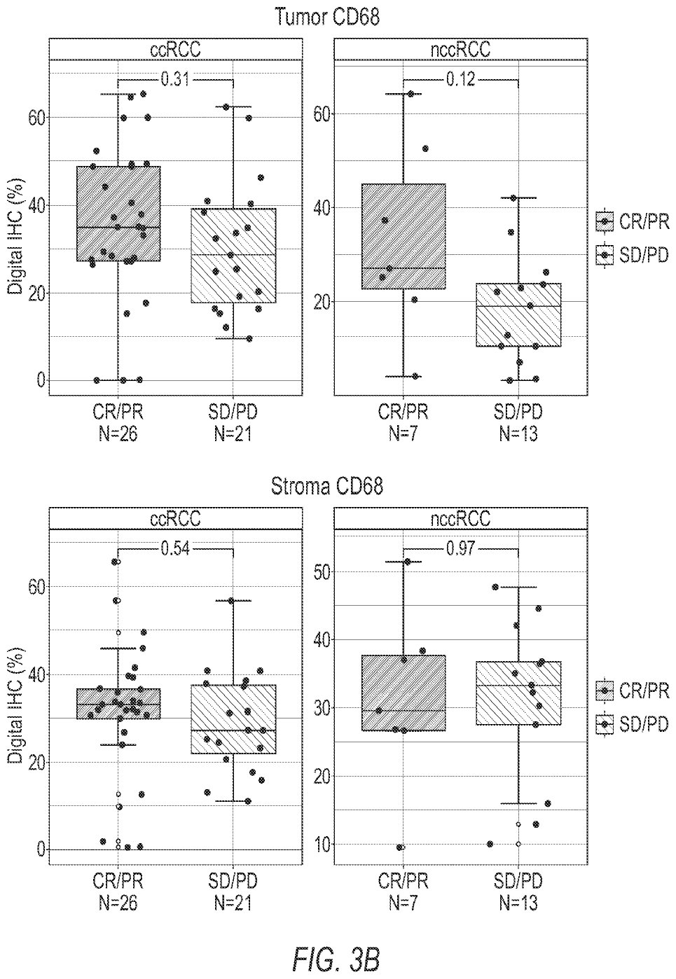

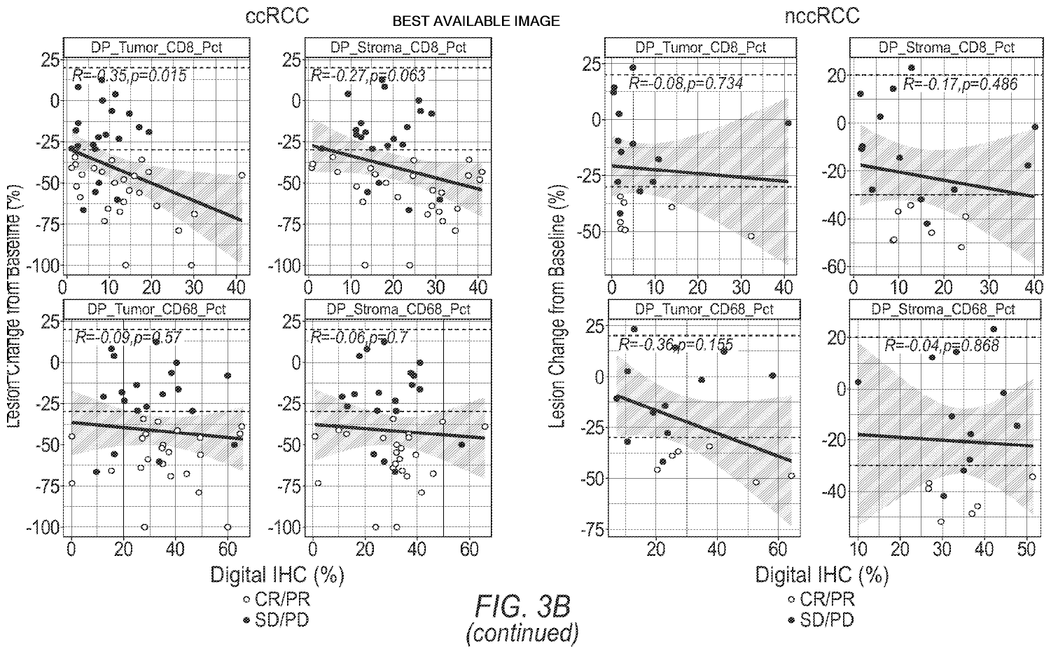

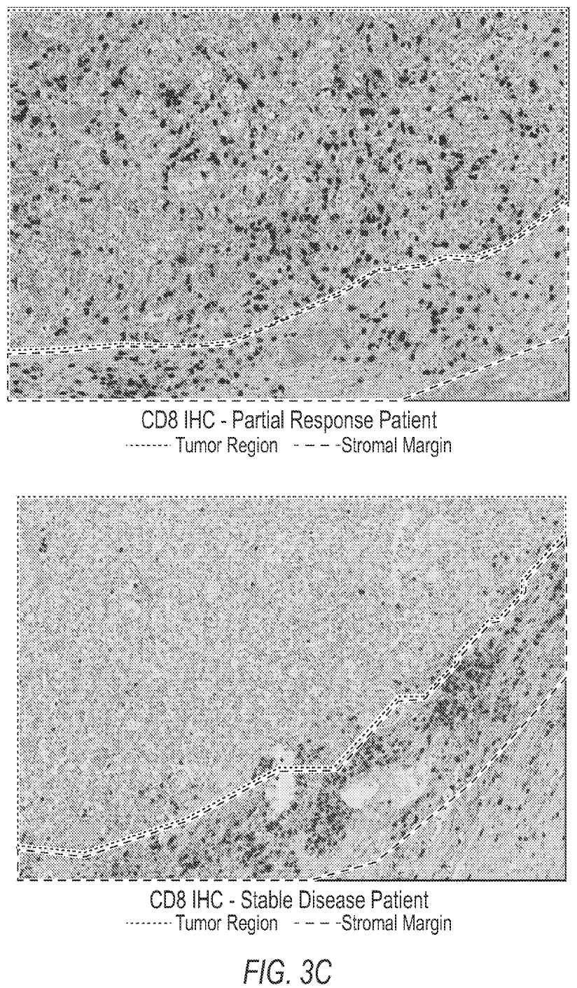

Archived collected within 2 years of enrollment, or fresh tumor tissue was obtained prior to dosing. In baseline tumor tissue, PD-L1 positive cells as determined by IHC using SP142 anti PD-L1 antibody (IC ≥1%) were evaluated for correlation to response and lesion change for ccRCC and nccRCC subjects (FIG. 2A). A greater number of ccRCC PD-L1 positive responders (53.6%) vs non responders (9.1%) was observed (p=0.001). For nccRCC subjects, no PD-L1 positive responders and 21% of non-responders were PD-L1 positive but there was no correlation with response. A larger decrement in lesion change correlated significantly with PD-L1 positive tumors (p >0.009) for ccRCC, and no significant correlation was observed for PD-L1 positive nccRCC tumors which appear to have lower decremental change in responders compared to non-responders. Moreover, 69.2% of PD-L1 immune cell (IC) positive tumors significantly correlated with responders (p=0.0003) and with best lesion change (p=0.0023) (FIG. 3A). Thus, PD-L1 positive immune cells are significantly associated with ORR response and lesion change from baseline in ccRCC patients. No correlation with PD-L1 IC was observed for nccRCC responders. Therefore, PD-L1 expression can be a useful biomarker to predict response and select patients, such as ccRCC patients to the treatment of cabozantinib in combination with atezolizumab.

Tumor tissue were evaluated for correlation to response and lesion change for CD8+ T cells, where the threshold for positive cells is ≥20% (FIG. 2B). 48.1% of ccRCC CD8+ responders and 14.3% of non-responders with CD8 positive T cells was observed (p=0.01). 14.3% of responders as compared to 21.4% of non-responders were CD8 positive for nccRCC subjects, but there was no significant association. Similar to the PD-L1 positive assessments, CD8+ tumors significantly correlated with greater lesion change in ccRCC (p>0.015) and did not associate with CD8+ tumors in responders or non-responders in nccRCC tumors. Thus, high frequency of CD8 T-cells cells is significantly associated with ORR response and lesion change from baseline in ccRCC patients. CD8 population therefore can be a useful biomarker to predict response and select patients, such as ccRCC patients to the treatment of cabozantinib in combination with atezolizumab.

PD-L1 positive and/or CD8+ tumors significantly correlated with 69.2% of ccRCC responders and 14.3% of non-responders (p=0.0003) but no correlation was found for nccRCC tumors.

Evaluation of the T and myeloid cell populations significantly correlated with ccRCC responders (p=0.04) and greater lesion change for the T cell hot (CD8high CD68high) and myeloid rich (CD8low CD68high) (p=0.052) cell populations as compared to immune low (CD8low CD68low) (p=0.029) (FIG. 2C). Thus, patients who were CD8 T-cell high and CD68 high were significantly associated with ORR response and lesion change from baseline in ccRCC cohort. No association was observed for these cell populations in nccRCC tumors.

Digital pathology analysis showed a significant percentage of CD8+ cells in tumor but no correlation was observed in stromal cells in ccRCC tumors (FIG. 2D). CD68 distribution was similar in both histological RCC subtypes (FIG. 3B). Representative IHC images of a partial responder and a stable disease subject are shown in FIG. 3C. Correlation of CD8 high ccRCC tumor (≥20%) with PFS was observed (p=0.02, HR 2.61 (1.16; 5.87), but not observed with nccRCC tumors. A similar association was observed when evaluating the digital pathology for CD8 (FIG. 2E). Thus, high frequency of CD8 T-cells cells is associated with PFS survival in ccRCC patients.

Collectively, these data indicates the pronounced immunogenic activity of ccRCC tumors as compared to nccRCC tumors, and the unexpected effect of cabozantinib in combination with atezolizumab for treating RCC such as ccRCC.

### Other Embodiments

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive.

The scope of the invention should, therefore, be determined not with reference to the above description, but should instead be determined with reference to the following appended claims, along with the full scope of equivalents to which such claims are entitled.

1. A method for predicting a treatment response of a subject having cancer to a therapy comprising cabozantinib in combination with an immune checkpoint inhibitor or a cancer vaccine, the method comprising:
a. measuring the myeloid cell and T cell populations in a tumor tissue sample from the subject; and
b. associating the myeloid cell and T cell populations with the treatment response of the subject based on the relative abundance/percentage of myeloid cell population and T cell population.

2. A method for identifying a subject having cancer as a candidate for treatment with a therapy comprising cabozantinib in combination with an immune checkpoint inhibitor or a cancer vaccine, the method comprising:
analyzing the myeloid cell and T cell populations in a tumor tissue sample from the subject, wherein the relative abundance/percentage of myeloid cell population and T cell population indicates the subject may benefit from the treatment with cabozantinib in combination with an immune checkpoint inhibitor or a cancer vaccine.

3. A method for identifying a subject having cancer that has an increased benefit from a treatment comprising cabozantinib in combination with an immune checkpoint inhibitor or a cancer vaccine, the method comprising:
analyzing the myeloid cell and T cell populations in a tumor tissue sample from the subject, wherein the relative abundance/percentage of myeloid cell population and T cell population is indicative of the subject may benefit from the treatment.

4. A method of treating cancer in a subject, the method comprising:
a. analyzing the myeloid cell and T cell populations in a tumor tissue sample from the subject; wherein the relative abundance/percentage of myeloid cell population and T cell population is indicative of the subject may benefit from the treatment; and
b. treating the subject in need of the treatment with a combination therapy comprising cabozantinib in combination with an immune checkpoint inhibitor or a cancer vaccine.

5. The method of any one of claims 1-4, wherein the immune checkpoint inhibitor is an inhibitor of PD-1 or PD-L1.

6. The method of any one of claims 1-5, wherein the immune checkpoint inhibitor is an anti-PD-1 antibody or anti-PD-L1 antibody.

7. The method of any one of claims 1-6, wherein the immune checkpoint inhibitor is an anti-PD-1 monoclonal antibody, an anti-PD-1 bispecific antibody, an anti-PD-L1 monoclonal antibody, or an anti-PD-L1 bispecific antibody.

8. The method of any one of claims 1-7, wherein the immune checkpoint inhibitor is ipilimumab, nivolumab, pembrolizumab, cemiplimab, durvalumab, atezolizumab or avelumab.

9. The method of any one of claims 1-8, wherein the myeloid cell is CD68 and the T cell is CD8.

10. The method of any one of claims 1-9, wherein the tumor tissue sample has high levels of CD68 and CD8 cells; high level of CD68 and low level of CD8 cells; or low levels of CD68 and CD8 cells.

11. The method of claim 10, wherein the tumor tissue sample has high levels of CD68 and CD8 cells.

12. The method of claim 11, wherein the tumor tissue sample has 20% or greater of CD68 and CD8 cells.

13. A method of treating a subject having a solid tumor with cabozantinib in combination with an immune checkpoint inhibitor therapy comprising:
a. determining whether the solid tumor comprises myeloid CD68 cells and cytotoxic CD8 T-cells; and
b. administering one or more doses of cabozantinib and one or more doses of immune checkpoint inhibitor therapy to the subject if the tumor comprises myeloid CD68 cells and cytotoxic CD8 T-cells, wherein the presence of myeloid CD68 cells and cytotoxic CD8 T-cells in the tumor indicates the responsiveness of the tumor to treatment with the checkpoint inhibitor therapy.

14. The method of any of claims 1-13, wherein the subject is human.

15. The method of claim 13, comprising administering one or more doses of cabozantinib and one or more doses of immune checkpoint inhibitor therapy to the subject if the tumor comprises high levels of myeloid CD68 cells and cytotoxic CD8 T-cells; high level of myeloid CD68 cells and low level of cytotoxic CD8 T-cells; or low levels of myeloid CD68 cells and cytotoxic CD8 T-cells.

16. The method of claim 15, comprising administering one or more doses of cabozantinib and one or more doses of immune checkpoint inhibitor therapy to the subject if the tumor comprises high levels of myeloid CD68 cells and cytotoxic CD8 T-cells.

17. The method of any one of claims 4-16, wherein cabozantinib is administered in a dosage from 20 to 60 mg.

18. The method of any one of claims 4-17, wherein cabozantinib is administered in a dosage of 40 or 60 mg.

19. The method of any one of claims 1-18, wherein the immune checkpoint inhibitor is atezolizumab.

20. The method of any one of claims 1-19, wherein the cancer is clear cell renal cell carcinoma.

21. Use of cabozantinib in combination with an immune checkpoint inhibitor or a cancer vaccine for the preparation of a medicament for the treatment of cancer in a subject, wherein the treatment comprises:
a. determining myeloid cell and T cell populations in a cancer sample from the subject, wherein the relative abundance/percentage of myeloid cell population and T cell population is indicative of the subject may benefit from the treatment; and
b. determining a therapeutically effective amount of cabozantinib for the treatment according to the relative abundance/percentage of myeloid cell population and T cell population.

22. The use of claim 21, wherein the cancer sample has high levels of myeloid CD68 cells and cytotoxic CD8 T-cells.

23. The use of claim 22, wherein the levels of myeloid CD68 cells and cytotoxic CD8 T-cells are 20% or greater.

24. The use of any one of claims 21-23, wherein the immune checkpoint inhibitor is atezolizumab.

25. The use of any one of claims 21-24, wherein the cancer is clear cell renal cell carcinoma.

26. A method for predicting a treatment response of a subject having cancer to a therapy comprising cabozantinib in combination with an immune checkpoint inhibitor or a cancer vaccine, the method comprising:
a. measuring T cell population and/or PD-L1 expression in a tumor tissue sample from the subject; and
b. associating the T cell population and/or PD-L1 expression with the treatment response of the subject based on the relative abundance/percentage of the T cell population and/or PD-L1 expression status.

27. A method for identifying a subject having cancer as a candidate for treatment with a therapy comprising cabozantinib in combination with an immune checkpoint inhibitor or a cancer vaccine, the method comprising:
analyzing T cell population and/or PD-L1 expression in a tumor tissue sample from the subject, wherein the the relative abundance/percentage of the T cell population and/or PD-L1 positive status indicates that the subject may benefit from the treatment with cabozantinib in combination with the immune checkpoint inhibitor or cancer vaccine.

28. A method of treating cancer in a subject, the method comprising:
a. analyzing T cell population and/or PD-L1 expression in a tumor tissue sample from the subject; wherein the relative abundance/percentage of T cell population and/or PD-L1 positive status is indicative that the subject may benefit from the treatment; and
b. treating the subject in need of the treatment with a combination therapy comprising cabozantinib in combination with an immune checkpoint inhibitor or a cancer vaccine.

29. The method of any one of claims 26-28, wherein the immune checkpoint inhibitor is an inhibitor of PD-1 or PD-L1.

30. The method of any one of claims 26-29, wherein the immune checkpoint inhibitor is an anti-PD-1 antibody or anti-PD-L1 antibody.

31. The method of any one of claims 26-30, wherein the immune checkpoint inhibitor is an anti-PD-1 monoclonal antibody, an anti-PD-1 bispecific antibody, an anti-PD-L1 monoclonal antibody, or an anti-PD-L1 bispecific antibody.

32. The method of any one of claims 26-31, wherein the immune checkpoint inhibitor is ipilimumab, nivolumab, pembrolizumab, cemiplimab, durvalumab, atezolizumab or avelumab.

33. The method of any one of claims 26-32, wherein the T cell is CD8 cells.

34. The method of any one of claims 26-33, wherein the tumor tissue sample has high levels of CD8 cells and/or PD-L1 positive immune cells.

35. A method of treating a subject having a solid tumor with cabozantinib in combination with an immune checkpoint inhibitor, the method comprising:
a. determining CD8 T cell level and/or PD-L1 expression in a tumor tissue sample from the subject; and
b. administering one or more doses of cabozantinib and one or more doses of the immune checkpoint inhibitor to the subject if the tumor comprises CD8 T cells and/or PD-L1 positive immune cells, wherein the presence of CD8 T cells and/or PD-L1 positive immune cells indicates the responsiveness of the tumor to treatment with the checkpoint inhibitor therapy.

36. The method of claim 35, comprising administering one or more doses of cabozantinib and one or more doses of immune checkpoint inhibitor therapy to the subject if the tumor comprises high level of CD8 T-cells.

37. The method of claim 35 or 36, comprising administering one or more doses of cabozantinib and one or more doses of immune checkpoint inhibitor therapy to the subject if the tumor comprises PD-L1 positive immune cells.

38. The method of any one of claims 26-37, wherein cabozantinib is administered in a dosage from 20 to 60 mg.

39. The method of claim 38, wherein cabozantinib is administered in a dosage of 40 or 60 mg.

40. The method of any one of claims 26-39, wherein the immune checkpoint inhibitor is atezolizumab.

41. The method of any one of claims 26-40, wherein the cancer is clear cell renal cell carcinoma.
